(19) United States
(12) Patent Application Publication
(10) Pub. No.: US 2025/0129115 A1
(43) Pub. Date: Apr. 24, 2025

(54) Methods Detecting Tertiary Amines and Compositions Related Thereto

(71) Applicant: Emory University, Atlanta, GA (US)

(72) Inventors: Monika Raj, Atlanta, GA (US); Benjamin Emenike, Atlanta, GA (US)

(21) Appl. No.: 18/694,335

(22) PCT Filed: Sep. 21, 2022
(86) PCT No.: PCT/US2022/076782

§ 371 Date: Mar. 21, 2024

Related U.S. Application Data
(60) Provisional application No. 63/246,695, filed on Sep. 21, 2021.
(60) Provisional application No. 63/280,802, filed on Nov. 18, 2021.
(60) Provisional application No. 63/401,372, filed on Aug. 26, 2022.

(51) Int. Cl. C07K 1/13; C07K 1/14

(52) U.S. Cl. CPC C07K 1/13; C07K 1/14

(57) ABSTRACT

This disclosure relates to methods and compositions for labeling, isolating, detecting, measuring, and purifying tertiary amines. In certain embodiments, the tertiary amine has a dimethyl amine such as in the case of dimethyl lysine, and N-terminal dimethyl amino groups in peptides. In certain embodiments, this disclosure relates to methods of labeling, isolating, detecting, measuring, and purifying compounds having tertiary amines, dimethyl lysines, or N-terminal dimethyl amines in proteins, or nucleic acids containing the same from a sample optionally utilizing solid supports.

### CROSS-REFERENCE TO RELATED APPLICATIONS

This application claims the benefit of U.S. Provisional Application No. 63/401,372 filed Aug. 26, 2022, U.S. Provisional Application No. 63/280,802 filed Nov. 18, 2021, and U.S. Provisional Application No. 63/246,695 filed Sep. 21, 2021. The entirety of each of these applications is hereby incorporated by reference for all purposes.

### STATEMENT REGARDING FEDERALLY SPONSORED RESEARCH OR DEVELOPMENT

This invention was made with government support under CHE-2108774 awarded by the National Science Foundation. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 21187PCT.xml. The XML file is 3 KB, was created on Sep. 20, 2022, and is being submitted electronically via the USPTO patent electronic filing system.

### BACKGROUND

Lysine methylation in a protein is a posttranslational modification (PTM) involved in the regulation of various biological processes. Different levels of mono-, di-, and tri-methylation of lysine may result in different functions and localization within a cell. Controlling PTM of lysine, e.g., to monomethyl lysine (Kme) or dimethyl lysine (Kme2), is reported in transcriptional activation and linked to numerous diseases and disorders such as heart disease, cancer, and diabetes. Methods to specifically detect the methylation status of lysine are limited because the addition of a methyl group(s) leads to minor alteration in physicochemical properties of proteins. Methylation of lysine does not typically add a substantial steric bulk or a significantly change hydrophobicity. Antibodies and other affinity reagents suffer from drawbacks. They are typically unable to completely detect all the methylation sites and unable to distinguish between different lysine methylation states (mono, di- or tri-). Another approach for detecting lysine methylation utilizes mass spectrometry (MS). However, MS analysis is limited as it does not directly confirm structural information and unable to detect low abundant lysine methylation PTMs. Thus, there is need to identify improved methods for detecting lysine methylation status.
Jones et al. report the oxidative coupling of tertiary amines. Synlett, 2012, 23, 159-162.
Michael North reports the oxidative synthesis of alpha-amino nitriles from tertiary amines. Angew Chem Int Ed, 2004, 43, 4126-4128.
Kolesinska et al. report transformation of tertiary amines into alkylating reagents by treatment with 2-chloro-4,6-dimethoxy-1,3,5-triazine. Polish J. Chem., 2008, 82, 2115-2123.
Sole et al. report sustainable triazine-based dehydro-condensation agents for amide synthesis. Molecules, 2021, 26, 191.

References cited herein are not an admission of prior art.

### SUMMARY

This disclosure relates to methods and compositions for labeling, isolating, detecting, measuring, and purifying tertiary amines. In certain embodiments, the tertiary amine has a dimethyl amine such as in the case of dimethyl lysine and N-terminal dimethyl amino groups in peptides. In certain embodiments, this disclosure relates to methods of labeling, isolating, detecting, measuring, and purifying compounds having tertiary amines, dimethyl lysines, or N-terminal dimethyl amines in proteins, or nucleic acids containing the same from a sample optionally utilizing solid supports.

In certain embodiments, this disclosure relates to methods of modifying a compound comprising contacting a tertiary amine compound having a N,N′-dimethyl amine group with a cyanide salt and an oxidizing agent under conditions such that a compound having a 2-(methylamino)acetonitrile group is formed in place of N,N′-dimethyl amine. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide or protein having a N,N′-dimethyl lysine amino acid.

In certain embodiments, this disclosure relates to methods of modifying a peptide comprising contacting a compound or peptide having an N-terminal N,N′-dimethyl amine group with a cyanide salt and an oxidizing agent under conditions such that a peptide substituted with a nitrile group is formed; and contacting the peptide substituted with a nitrile group with labeled 2-aminoethane-1-thiol providing a thiazolidine labeled compound. In certain embodiments, the oxidizing agent is 1-chloromethyl-4-fluoro-1,4-diazoniabicyclo[2.2.2]octane bis(tetrafluoroborate) (Selectfluor).

In certain embodiments, the label is an alkynyl group providing alkynyl labeled peptide. In certain embodiments, methods further comprise contacting the alkynyl labeled peptide with a solid surface conjugated to a triazene under conditions such that a triazole peptide is conjugated to the solid surface.

In certain embodiments, methods further comprise contacting the triazole peptide conjugated to the solid surface with an acid solution such that the purified peptide is cleaved from the solid surface providing purified peptide.

In certain embodiments, the method further comprises contacting the compound having a 2-(methylamino)acetonitrile group with labeled indole providing a N-((1H-indol-3-yl)methyl)-N-methylmethan-1-amine labeled compound.

In certain embodiments, the method further comprises contacting the compound having a 2-(methylamino)acetonitrile group with labeled furan providing N-((furan-2-yl)methyl)-N-methylmethan-1-amine labeled compound.

In certain embodiments, the method further comprises contacting the compound having a 2-(methylamino)acetonitrile group with labeled 1-methyl-1H-pyrrole providing a N-((1-methyl-1H-pyrrol-2-yl)methyl)-N-methylmethan-1-amine labeled compound.

In certain embodiments, the method further comprises contacting the compound having a 2-(methylamino)acetonitrile group with 3-ethynyl-1-methyl-1H-pyrrole providing a N-((3-ethynyl-1-methyl-1H-pyrrol-2-yl)methyl)-N-methylmethan-1-amine labeled compound.

In certain embodiments, the method further comprises contacting the N-((3-ethynyl-1-methyl-1H-pyrrol-2-yl)methyl)-N-methylmethan-1-amine labeled compound with a solid surface conjugated to a triazene under conditions such that a N-methyl-N-((1-methyl-3-(1,2,3-triazol-4-yl)-1H-pyrrol-2-yl)methyl) methan-1-amine compound is conjugated to the solid surface.

In certain embodiments, the label is biotin providing N-((1-methyl-1H-pyrrol-2-yl)methyl)-N-methylmethan-1-amine biotin labeled compound. In certain embodiments, the method further comprises contacting the N-((1-methyl-1H-pyrrol-2-yl)methyl)-N-methylmethan-1-amine biotin labeled compound with a solid surface conjugated to avidin or streptavidin under conditions such that N-((1-methyl-1H-pyrrol-2-yl)methyl)-N-methylmethan-1-amine biotin labeled compound is conjugated to the solid surface.

In certain embodiments, the method further comprises contacting the compound having a 2-(methylamino)acetonitrile group with labeled furan providing a N-((furan-2-yl)methyl)-N-methylmethan-1-amine labeled compound.

In certain embodiments, the method further comprises contacting the compound having a 2-(methylamino)acetonitrile group with 3-ethynylfuran providing a N-((3-ethynylfuran-pyrrol-2-yl)methyl)-N-methylmethan-1-amine labeled compound.

In certain embodiments, the method further comprises contacting the N-((3-ethynylfuran-2-yl)methyl)-N-methylmethan-1-amine labeled compound with a solid surface conjugated to a triazene under conditions such that a N-((3-(1,2,3-triazol-4-yl) furan-2-yl)methyl)-N-methylmethan-1-amine compound is conjugated to the solid surface.

In certain embodiments, the label is biotin providing N-((furan-2-yl)methyl)-N-methylmethan-1-amine biotin labeled compound. In certain embodiments, the method further comprises contacting the N-((furan-2-yl)methyl)-N-methylmethan-1-amine biotin labeled compound with a solid surface conjugated to avidin or streptavidin under conditions such that N-((furan-2-yl)methyl)-N-methylmethan-1-amine biotin labeled compound is conjugated to the solid surface.

In certain embodiments, the method further comprises contacting the compound having a 2-(methylamino)acetonitrile group with labeled ketone or propan-2-one (acetone) providing a 4-(dimethylamino) butan-2-one labeled compound.

In certain embodiments, the method further comprises contacting the compound having a 2-(methylamino)acetonitrile group with pent-4-yn-2-one providing a 1-(dimethylamino) hex-5-yn-3-one labeled compound.

In certain embodiments, the method further comprises contacting the 1-(dimethylamino) hex-5-yn-3-one labeled compound with a solid surface conjugated to a triazene under conditions such that a 4-(dimethylamino)-1-(1,2,3-triazol-4-yl) butan-2-one compound is conjugated to the solid surface.

In certain embodiments, the label is biotin providing propan-2-one (acetone) biotin labeled compound. In certain embodiments, the method further comprises contacting the propan-2-one (acetone) biotin labeled compound with a solid surface conjugated to avidin or streptavidin under conditions such that 4-(dimethylamino) butan-2-one biotin labeled compound is conjugated to the solid surface.

In certain embodiments, the method further comprises contacting the compound having a 2-(methylamino)acetonitrile group with allyltrimethylsilane providing a compound having an N-methyl-N-methylbut-3-en-1-amine group.

In certain embodiments, the method further comprises contacting the compound having the N-methyl-N-methylbut-3-en-1-amine group with ozone providing a compound having a 3-(dimethylamino)propanal group.

In certain embodiments, the method further comprises contacting the compound having a 3-(dimethylamino)propanal group with a solid surface comprising a primary amine, alkyl amine, aniline, or hydrazine group under conditions such that a N-(3-(azaneylidene)propyl)-N-methylbutan-1-amine compound is conjugated to the solid surface having a hydrazine or imine Schiff base linkage.

In certain embodiments, this disclosure relates to methods of modifying a compound comprising contacting a tertiary amine compound having a N,N′-dimethyl amine group with labeled indole and an oxidizing agent under conditions such that N-((1H-indol-3-yl)methyl)-N-methylamine labeled compound is formed in place of the N,N′-dimethyl amine group. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide having a N,N′-dimethyl lysine amino acid.

In certain embodiments, the label is an alkynyl group providing N-((1H-indol-3-yl)methyl)-N-methylamine alkynyl labeled compound. In certain embodiments, the method further comprises contacting the N-((1H-indol-3-yl)methyl)-N-methylamine alkynyl labeled compound with a solid surface conjugated to a triazene under conditions such that a N-((1H-indol-3-yl)methyl)-N-methylamine triazole compound is formed on the solid surface. In certain embodiments, the method further comprises contacting the N-((1H-indol-3-yl)methyl)-N-methylamine triazole compound conjugated to the solid surface with an acid solution such that the compound is cleaved from the solid surface.

In certain embodiments, the label is biotin providing N-((1H-indol-3-yl)methyl)-N-methylamine biotin labeled compound. In certain embodiments, the method further comprises contacting the N-((1H-indol-3-yl)methyl)-N-methylamine biotin labeled compound with a solid surface conjugated to avidin or streptavidin providing a N-((1H-indol-3-yl)methyl)-N-methylamine biotin compound conjugated to the solid surface.

In certain embodiments, this disclosure relates to methods of modifying a compound comprising contacting a tertiary amine compound having a N,N′-dimethyl amine group with labeled furan and an oxidizing agent under conditions such that N-((furan-2-yl)methyl)-N-methyl methyl-1-amine labeled compound is formed in place of the N,N′-dimethyl amine group. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide having a N,N′-dimethyl lysine amino acid.

In certain embodiments, this disclosure relates to methods of modifying a compound comprising contacting a tertiary amine compound having a N,N′-dimethyl amine group with labeled pyrrole and an oxidizing agent under conditions such that N-((1H-pyrrol-2-yl)methyl)-N-methylmeth-1-amine labeled compound is formed in place of the N,N′-dimethyl amine group. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide having a N,N′-dimethyl lysine amino acid.

In certain embodiments, this disclosure relates to methods of modifying a compound comprising contacting a tertiary amine compound having a N,N′-dimethyl amine group with allyltrimethylsilane or allytrialkylsilane and an oxidizing agent under conditions such that N-methyl-N-methybut-3-en-1-amine labeled compound is formed in place of the N,N′-dimethyl amine group. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide having a N,N′-dimethyl lysine amino acid.

In certain embodiments, this disclosure relates to methods of modifying a compound comprising contacting a tertiary amine compound having a N,N′-dimethyl amine group with a labeled ketone and an oxidizing agent under conditions such that N-(dimethylamino) butan-2-one labeled compound is formed in place of the N,N′-dimethyl amine group. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide having a N,N′-dimethyl lysine amino acid.

In certain embodiments, this disclosure relates to methods of modifying a compound comprising contacting a tertiary amine compound having a N,N′-dimethyl amine group with 4-dimethylamino pyridine, pyridine, or pyridine having a para substituted tertiary amine and an oxidizing agent under conditions such that an aldehyde group is formed in place of N,N′-dimethyl amine, e.g., converting dimethyl lysine side chain into butyraldehyde. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide or protein having a N,N′-dimethyl lysine amino acid.

In certain embodiments, the method further comprises contacting the compound having the aldehyde group with a primary amine, hydrazine, alkyl amine, or aniline group providing a compound having a hydrazine or imine Schiff base linkage, e.g., 1-butylidene-2-methylhydrazine compound or N-methylbutan-1-imine compound optionally labeled.

In certain embodiments, the method further comprises contacting the compound having aldehyde group with a solid surface comprising a primary amine group, alkyl amine, aniline, or hydrazine group under conditions such that a compound having a hydrazine or imine Schiff base linkage, e.g., 1-butylidene-2-methylhydrazine compound or N-methylbutan-1-imine compound is conjugated to the solid surface.

In certain embodiments, the method further comprises contacting the compound having aldehyde group with a solid surface comprising primary amine group, alkyl amine, aniline, or hydrazine group under conditions such that a labeled compound having a hydrazine or imine Schiff base linkage is formed. In certain embodiments, the labeled compound is an alkynyl labeled compound providing 1-butylidene-2-(prop-2-yn-1-yl) hydrazine compound.

In certain embodiments, the method further comprises contacting the alkynyl labeled compound with a triazene providing a triazole labeled compound. In certain embodiments, the triazene is conjugated to a solid surface providing a triazole linkage to the compound.

In certain embodiments, this disclosure relates to compositions comprising reagents and solid surfaces/supports used for making modifications disclosed herein, including intermediates, and products resulting therefrom.

### BRIEF DESCRIPTION OF THE SEVERAL VIEWS OF THE DRAWINGS

FIG. 1A illustrates tertiary amine coupling by oxidation (TACO) using Selectfluor™ as the oxidizing agent followed by nucleophilic addition.

FIG. 1B shows data indicating that the use of Selectfluor™ and NaCN in the reaction is highly electivity for Kme2 modification as quantified by LC/MS.

FIG. 2A illustrates selective modification of oxidized Kme2 with different nucleophiles such as phenylacetylene, indoles, acetones, furans, and pyrroles.

FIG. 2B illustrates the chemical names of intermediates using indole or an alkynyl as a nucleophile in TACO of tertiary amines.

FIG. 2C illustrates the chemical names of intermediates using 1-methyl-1H-pyrrole as a nucleophile in TACO of tertiary amines.

FIG. 2D illustrates the chemical names of intermediates using allytrimethylsilane in TACO of tertiary amines.

FIG. 2E illustrates the chemical names of intermediates using alkynyl labeled acetone as a nucleophile in TACO of tertiary amines.

FIG. 2F illustrates the chemical names of intermediates using labeled furan as a nucleophile in TACO of tertiary amines.

FIG. 3A illustrates labeling of peptides by oxidation of dimethyl lysine with 4-dimethylamino pyridine (DMAP) providing an aldehyde and coupling the aldehyde with a hydrazine or hydrazine label, e.g., alkynyl label.

FIG. 3B illustrates the chemical names of intermediates using the aldehyde and labeled hydrazine.

FIG. 4A illustrates the chemical modification of N-terminal dimethyl amines resulting in the formation of thiazolidine derivatives.

FIG. 4B illustrates the chemical names of intermediates using labeled thiazolidine derivatives.

FIG. 4C illustrates plausible intermediates for thiazolidine formation through an alpha keto amide. It is not intended that certain embodiments of this disclosure be limited by any particular mechanism.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An embodiment of this disclosure indicates that it is an example and not necessarily limited to such example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

The terms “protein,” “peptide,” and “polypeptide” refer to compounds comprising amino acids joined via peptide bonds and are used interchangeably. As used herein, where “amino acid sequence” is recited herein to refer to an amino acid sequence of a protein molecule. An “amino acid sequence” can be deduced from the nucleic acid sequence encoding the protein. However, terms such as “peptide” or “protein” are not meant to limit be limited to natural amino acids, and it is contemplated that the terms include natural proteins and modified forms and non-naturally occurring amino acids and modifications such as, substitutions, glycosylations, and addition of hydrophilic or lipophilic moieties. In certain embodiments, it is contemplated that the peptide comprises more than three, four, five, six, seven, eight, nine or ten amino acids.

The term “nucleobase polymer” refers to a polymer comprising nitrogen containing aromatic or heterocyclic bases that bind to naturally occurring nucleic acids through hydrogen bonding otherwise known as base pairing. A typical nucleobase polymer is a nucleic acid, RNA, DNA, or chemically modified form thereof. A nucleobase polymer may contain DNA or RNA or a combination of DNA or RNA nucleotides or may be single or double stranded or both, e.g., they may contain overhangs, hairpins, bends, etc. Nucleobase polymers may contain naturally occurring or synthetically modified bases and backbones.

As used herein, the term “conjugated” refers to linking molecular entities through covalent bonds, or by other specific binding interactions, such as due to hydrogen bonding and other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

A “linking group” refers to any variety of molecular arrangements that can be used to bridge or conjugate molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)-, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRn—, (C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups, alkoxyalkyl, and aromatic groups.

The term “specific binding agent” refers to a molecule, such as a proteinaceous molecule, that binds a target molecule with a greater affinity than other random molecules or proteins. Examples of specific binding agents include antibodies that bind an epitope of an antigen or a receptor which binds a ligand. “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

As used herein, the term “ligand” refers to any organic molecule, i.e., substantially comprised of carbon, hydrogen, and oxygen, that specifically binds to a “receptor.” Receptors are organic molecules typically found on the surface of a cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin protein may bind the glycan. As the glycan is typically smaller and surrounded by the lectin protein during binding, it may be considered a ligand even though it is a receptor of the lectin binding signal on the cell surface. An antibody may be a receptor, and the epitope may be considered the ligand. In certain embodiments, a ligand is contemplated to be a compound that has a molecular weight of less than 500 or 1,000. In certain embodiments, a receptor is contemplated to be a protein-based compound that has a molecular weight of greater than 1,000, 2,000 or 5,000. In any of the embodiments disclosed herein the position of a ligand and a receptor may be switched.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a peptide “label” refers to incorporation of a heterologous polypeptide in the peptide, wherein the heterologous sequence can be identified by a specific binding agent, antibody, or bind to a metal such as nickel/nitrilotriacetic acid, e.g., a poly-histidine sequence. Specific binding agents and metals can be conjugated to solid surfaces to facilitate purification methods. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels may be attached by spacer arms of various lengths to reduce potential steric hindrance.

In certain contexts, an “antibody” refers to a protein-based molecule that is naturally produced by animals in response to the presence of a protein or other molecule or that is not recognized by the animal's immune system to be a “self” molecule, i.e., recognized by the animal to be a foreign molecule and an antigen to the antibody. The immune system of the animal will create an antibody to specifically bind the antigen, and thereby targeting the antigen for elimination or degradation. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, as used herein the term “antibody” is intended to include natural antibodies, monoclonal antibody, or non-naturally produced synthetic antibodies, and binding fragments, such as single chain binding fragments. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

Hydrophilic polymers contain polar or charged functional groups, rendering them soluble in water. Examples include polyethylene glycol, polylactides, polyglycolide, poly(ε-caprolactone), poly(2-methoxyethyl acrylate), poly(tetrahydrofurfuryl acrylate), poly(2-methacryloyloxyethyl phosphorylcholine), poly(p-dioxanone), poly(serine methacrylate), poly[oligo(ethylene glycol) vinyl ether], poly{[2-(methacryloyloxy)ethyl], copolymers of ethylene glycol and propylene glycol, poly(oxyethylated polyol), poly(olefmic alcohol), poly(vinylpyrrolidone), poly(hydroxyalkylmethacrylamide), poly(hydroxyalkylmethacrylate), poly(saccharides), poly(alpha-hydroxy acid), and poly(vinyl alcohol). “PEG,” “polyethylene glycol” and “poly(ethylene glycol)” refers to water-soluble poly(ethylene oxide). Typically, PEGs comprise the following structure “—(OCH₂CH₂)n-” where (n) is 2 to 4000.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom, replacing an amino group with a hydroxyl group, replacing a nitrogen with a protonated carbon (CH) in an aromatic ring, replacing a bridging amino group (—NH—) with an oxy group (—O—), or vice versa. A derivative may be a polypeptide variant. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRaNRb, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkylthio” refers to an alkyl group as defined above with the indicated number of carbon atoms attached through a sulfur bridge. An example of an alkylthio is methylthio, (i.e., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above with the indicated number of carbon atoms attached through an amino bridge. An example of an alkylamino is methylamino, (i.e., —NH—CH₃).

“Alkanoyl” refers to an alkyl as defined above with the indicated number of carbon atoms attached through a carbonyl bride (i.e., —(C═O)alkyl).

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

The terms “pyridine having a para substituted tertiary amine” refers to 4-dimethylaminopyridine which is optionally substituted or a derivative, e.g., the methyl groups and/or one or both hydrogen in the meta position on the pyridine ring and the attached atoms may for a ring. Examples include 4-dimethylaminopyridine, N,N,2-trimethylpyridin-4-amine, 4-(pyrrolidin-1-yl)pyridine, 2-methyl-4-(pyrrolidin-1-yl)pyridine, and 1-methyl-2,3-dihydro-1H-pyrrolo[3,2-c]pyridine. Numerous other examples are known. See e.g., Spivey et al. J. Chem. Soc., Perkin Trans. 1, 2001, 1785-1794 and Dalaigh et al. Org. Biomol. Chem., 2006, 4, 2785-2793.

### Methods

This disclosure relates to methods and compositions for labeling, isolating, detecting, measuring, and purifying tertiary amines. In certain embodiments, the tertiary amine is dimethyl amine such as in the case of dimethyl lysine and peptides containing dimethyl lysine or an N-terminal dimethyl amine group. In certain embodiments, this disclosure relates to methods of labeling, isolating, detecting, measuring, and purifying compounds having tertiary amine, dimethyl lysines, N-terminal dimethyl amines on peptides, proteins, or nucleic acids containing the same from a sample utilizing solid supports.

In certain embodiments, this disclosure relates to methods of modifying a compound comprising contacting a tertiary amine compound having a N,N′-dimethyl amine group with a cyanide salt and an oxidizing agent under conditions such that a compound having a 2-(methylamino)acetonitrile group is formed in place of N,N′-dimethyl amine. In certain embodiments, the oxidizing agent is 1-chloromethyl-4-fluoro-1,4-diazoniabicyclo[2.2.2]octane bis(tetrafluoroborate) (Selectfluor). In certain embodiments, the compound is a peptide having a N,N′-dimethyl lysine amino acid or an N-terminal dimethyl amine.

In certain embodiments, this disclosure relates to methods of modifying a compound comprising contacting a tertiary amine compound having a N,N′-dimethyl amine group with a cyanide salt and an oxidizing agent under conditions such that a compound having a 2-(methylamino)acetonitrile group is formed in place of N,N′-dimethyl amine. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide or protein having a N,N′-dimethyl lysine amino acid.

In certain embodiments, the method further comprises contacting the compound having a 2-(methylamino)acetonitrile group with labeled 1-methyl-1H-pyrrole providing a N-((1-methyl-1H-pyrrol-2-yl)methyl)-N-methylmethan-1-amine labeled compound.

In certain embodiments, the method further comprises contacting the compound having a 2-(methylamino)acetonitrile group with 3-ethynyl-1-methyl-1H-pyrrole providing a N-((3-ethynyl-1-methyl-1H-pyrrol-2-yl)methyl)-N-methylmethan-1-amine labeled compound.

In certain embodiments, the method further comprises contacting the N-((3-ethynyl-1-methyl-1H-pyrrol-2-yl)methyl)-N-methylmethan-1-amine labeled compound with a solid surface conjugated to a triazene under conditions such that a N-methyl-N-((1-methyl-3-(1,2,3-triazol-4-yl)-1H-pyrrol-2-yl)methyl) methan-1-amine compound is conjugated to the solid surface.

In certain embodiments, the label is biotin providing N-((1-methyl-1H-pyrrol-2-yl)methyl)-N-methylmethan-1-amine biotin labeled compound. In certain embodiments, the method further comprises contacting the N-((1-methyl-1H-pyrrol-2-yl)methyl)-N-methylmethan-1-amine biotin labeled compound with a solid surface conjugated to avidin or streptavidin under conditions such that N-((1-methyl-1H-pyrrol-2-yl)methyl)-N-methylmethan-1-amine biotin labeled compound is conjugated to the solid surface.

In certain embodiments, the method further comprises contacting the compound having a 2-(methylamino)acetonitrile group with labeled furan providing a N-((furan-2-yl)methyl)-N-methylmethan-1-amine labeled compound.

In certain embodiments, the method further comprises contacting the compound having a 2-(methylamino)acetonitrile group with 3-ethynylfuran providing a N-((3-ethynylfuran-pyrrol-2-yl)methyl)-N-methylmethan-1-amine labeled compound.

In certain embodiments, the method further comprises contacting the N-((3-ethynylfuran-2-yl)methyl)-N-methylmethan-1-amine labeled compound with a solid surface conjugated to a triazene under conditions such that a N-((3-(1,2,3-triazol-4-yl) furan-2-yl)methyl)-N-methylmethan-1-amine compound is conjugated to the solid surface.

In certain embodiments, the label is biotin providing N-((furan-2-yl)methyl)-N-methylmethan-1-amine biotin labeled compound. In certain embodiments, the method further comprises contacting the N-((furan-2-yl)methyl)-N-methylmethan-1-amine biotin labeled compound with a solid surface conjugated to avidin or streptavidin under conditions such that N-((furan-2-yl)methyl)-N-methylmethan-1-amine biotin labeled compound is conjugated to the solid surface.

In certain embodiments, the method further comprises contacting the compound having a 2-(methylamino)acetonitrile group with labeled ketone or propan-2-one (acetone) providing a 4-(dimethylamino) butan-2-one labeled compound.

In certain embodiments, the method further comprises contacting the compound having a 2-(methylamino)acetonitrile group with pent-4-yn-2-one providing a 1-(dimethylamino) hex-5-yn-3-one labeled compound.

In certain embodiments, the method further comprises contacting the 1-(dimethylamino) hex-5-yn-3-one labeled compound with a solid surface conjugated to a triazene under conditions such that a 4-(dimethylamino)-1-(1,2,3-triazol-4-yl) butan-2-one compound is conjugated to the solid surface.

In certain embodiments, the label is biotin providing propan-2-one (acetone) biotin labeled compound. In certain embodiments, the method further comprises contacting the propan-2-one (acetone) biotin labeled compound with a solid surface conjugated to avidin or streptavidin under conditions such that 4-(dimethylamino) butan-2-one biotin labeled compound is conjugated to the solid surface.

In certain embodiments, the method further comprises contacting the compound having a 2-(methylamino)acetonitrile group with allyltrimethylsilane providing a compound having an N-methyl-N-methylbut-3-en-1-amine group.

In certain embodiments, the method further comprises contacting the compound having the N-methyl-N-methylbut-3-en-1-amine group with ozone providing a compound having a 3-(dimethylamino)propanal group.

In certain embodiments, the method further comprises contacting the compound having a 3-(dimethylamino)propanal group with a solid surface comprising a primary amine group under conditions such that a N-(3-(azaneylidene)propyl)-N-methylbutan-1-amine compound is conjugated to the solid surface.

In certain embodiments, this disclosure relates to methods of modifying a compound comprising contacting a tertiary amine compound having a N,N′-dimethyl amine group with labeled indole and an oxidizing agent under conditions such that N-((1H-indol-3-yl)methyl)-N-methylamine labeled compound is formed in place of the N,N′-dimethyl amine group. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide having a N,N′-dimethyl lysine amino acid.

In certain embodiments, the label is an alkynyl group providing a N-((1H-indol-3-yl)methyl)-N-methylamine alkynyl labeled compound. In certain embodiments, the method further comprises contacting the N-((1H-indol-3-yl)methyl)-N-methylamine alkynyl labeled compound with a solid surface conjugated to a triazene under conditions such that a N-((1H-indol-3-yl)methyl)-N-methylamine triazole compound is formed on the solid surface. In certain embodiments, the method further comprises contacting the N-((1H-indol-3-yl)methyl)-N-methylamine triazole compound conjugated to the solid surface with an acid solution such that the compound is cleaved from the solid surface.

In certain embodiments, the label is biotin providing a N-((1H-indol-3-yl)methyl)-N-methylamine biotin labeled compound. In certain embodiments, the method further comprises contacting the N-((1H-indol-3-yl)methyl)-N-methylamine biotin labeled compound with a solid surface conjugated to avidin or streptavidin providing a N-((1H-indol-3-yl)methyl)-N-methylamine biotin compound conjugated to the solid surface.

In certain embodiments, this disclosure relates to methods of modifying a compound comprising contacting a tertiary amine compound having a N,N′-dimethyl amine group with labeled pyrrole and an oxidizing agent under conditions such that N-((1H-pyrrol-2-yl)methyl)-N-methylmeth-1-amine labeled compound is formed in place of the N,N′-dimethyl amine group. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide having a N,N′-dimethyl lysine amino acid.

In certain embodiments, this disclosure relates to methods of modifying a compound comprising contacting a tertiary amine compound having a N,N′-dimethyl amine group with allyltrimethylsilane or allytrialkylsilane and an oxidizing agent under conditions such that a N-methyl-N-methybut-3-en-1-amine labeled compound is formed in place of the N,N′-dimethyl amine group. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide having a N,N′-dimethyl lysine amino acid.

In certain embodiments, this disclosure relates to methods of modifying a compound comprising contacting a tertiary amine compound having a N,N′-dimethyl amine group with labeled indole and an oxidizing agent under conditions such that a N-((1H-indol-3-yl)methyl)-N-methylamine labeled compound is formed in place of the N,N′-dimethyl amine group. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide having a N,N′-dimethyl lysine amino acid.

In certain embodiments, the label is an alkynyl group providing a N-((1H-indol-3-yl)methyl)-N-methylamine alkynyl labeled compound. In certain embodiments, the method further comprises contacting the N-((1H-indol-3-yl)methyl)-N-methylamine alkynyl labeled compound with a solid surface conjugated to a triazene under conditions such that a N-((1H-indol-3-yl)methyl)-N-methylamine triazole compound is formed on the solid surface. In certain embodiments, the method further comprises contacting the N-((1H-indol-3-yl)methyl)-N-methylamine triazole compound conjugated to the solid surface with an acid solution such that the compound is cleaved from the solid surface.

In certain embodiments, the label is biotin providing a N-((1H-indol-3-yl)methyl)-N-methylamine biotin labeled compound. In certain embodiments, the method further comprises contacting the N-((1H-indol-3-yl)methyl)-N-methylamine biotin labeled compound with a solid surface conjugated to avidin or streptavidin providing a N-((1H-indol-3-yl)methyl)-N-methylamine biotin compound conjugated to the solid surface.

In certain embodiments, the label is a ligand providing a N-((1H-indol-3-yl)methyl)-N-methylamine ligand labeled compound. In certain embodiments, the method further comprises contacting the N-((1H-indol-3-yl)methyl)-N-methylamine ligand labeled compound with a solid surface conjugated to a receptor providing a N-((1H-indol-3-yl)methyl)-N-methylamine ligand compound conjugated to the solid surface.

In certain embodiments, the method further comprises contacting the compound having a 2-(methylamino)acetonitrile group with labeled 1-methyl-1H-pyrrole providing a N-((1-methyl-1H-pyrrol-2-yl)methyl)-N-methylmethan-1-amine labeled compound.

In certain embodiments, the method further comprises contacting the compound having a 2-(methylamino)acetonitrile group with 3-ethynyl-1-methyl-1H-pyrrole providing a N-((3-ethynyl-1-methyl-1H-pyrrol-2-yl)methyl)-N-methylmethan-1-amine labeled compound.

In certain embodiments, the method further comprises contacting the N-((3-ethynyl-1-methyl-1H-pyrrol-2-yl)methyl)-N-methylmethan-1-amine labeled compound with a solid surface conjugated to a triazene under conditions such that a N-methyl-N-((1-methyl-3-(1,2,3-triazol-4-yl)-1H-pyrrol-2-yl)methyl) methan-1-amine compound is conjugated to the solid surface.

In certain embodiments, the label is a ligand providing a N-((1-methyl-1H-pyrrol-2-yl)methyl)-N-methylmethan-1-amine ligand labeled compound. In certain embodiments, the method further comprises contacting the N-((1-methyl-1H-pyrrol-2-yl)methyl)-N-methylmethan-1-amine ligand labeled compound with a solid surface conjugated to receptor under conditions such that a N-methyl-N-((1-methyl-1H-pyrrol-2-yl)methyl) methan-1-amine ligand labeled compound is conjugated to the solid surface.

In certain embodiments, the label is biotin providing a N-((1-methyl-1H-pyrrol-2-yl)methyl)-N-methylmethan-1-amine biotin labeled compound. In certain embodiments, the method further comprises contacting the N-((1-methyl-1H-pyrrol-2-yl)methyl)-N-methylmethan-1-amine biotin labeled compound with a solid surface conjugated to avidin or streptavidin under conditions such that a N-methyl-N-((1-methyl-1H-pyrrol-2-yl)methyl) methan-1-amine biotin labeled compound is conjugated to the solid surface.

In certain embodiments, the method further comprises contacting the compound having a 2-(methylamino)acetonitrile group with labeled propan-2-one (acetone) providing a 4-(dimethylamino) butan-2-one labeled compound.

In certain embodiments, the method further comprises contacting the compound having a 2-(methylamino)acetonitrile group with pent-4-yn-2-one providing a 1-(dimethylamino) hex-5-yn-3-one labeled compound.

In certain embodiments, the method further comprises contacting the 1-(dimethylamino) hex-5-yn-3-one labeled compound with a solid surface conjugated to a triazene under conditions such that a 4-(dimethylamino)-1-(1,2,3-triazol-4-yl) butan-2-one compound is conjugated to the solid surface.

In certain embodiments, the label is a ligand providing a propan-2-one (acetone) ligand labeled compound. In certain embodiments, the method further comprises contacting the propan-2-one (acetone) ligand labeled compound with a solid surface conjugated to a receptor under conditions such that 4-(dimethylamino) butan-2-one ligand labeled compound is conjugated to the solid surface.

In certain embodiments, the label is biotin providing a propan-2-one (acetone) biotin labeled compound. In certain embodiments, the method further comprises contacting the propan-2-one (acetone) biotin labeled compound with a solid surface conjugated to avidin or streptavidin under conditions such that a 4-(dimethylamino)butan-2-one biotin labeled compound is conjugated to the solid surface.

In certain embodiments, the method further comprises contacting the compound having a 2-(methylamino)acetonitrile group with allyltrimethylsilane providing a compound having a N-methyl-N-methylbut-3-en-1-amine group.

In certain embodiments, the method further comprises contacting the compound having the N-methyl-N-methylbut-3-en-1-amine group with ozone providing a compound having a 3-(dimethylamino)propanal group.

In certain embodiments, the method further comprises contacting the compound having a 3-(dimethylamino)propanal group with a solid surface comprising a primary amine group under conditions such that a N-(3-(azaneylidene)propyl)-N-methylbutan-1-amine compound is conjugated to the solid surface.

In certain embodiments, this disclosure relates to methods of modifying a peptide comprising N,N′-dimethyl lysine comprising contacting a peptide having a N,N′-dimethyl lysine amino acid with a cyanide salt and an oxidizing agent under conditions such that 2-(methyl(butyl)amino)acetonitrile is formed in place of N,N′-dimethyl lysine. In certain embodiments, the oxidizing agent is 1-chloromethyl-4-fluoro-1,4-diazoniabicyclo[2.2.2]octane bis(tetrafluoroborate) (Selectfluor™). In certain embodiments, the method further comprises contacting the 2-(methyl(butyl)amino)acetonitrile with labeled 1-methyl-1H-pyrrole providing a N-((1-methyl-1H-pyrrol-2-yl)methyl)-N-methylbutan-1-amine labeled peptide.

In certain embodiments, the method further comprises contacting the peptide having a 2-(methylamino)acetonitrile group with 3-ethynyl-1-methyl-1H-pyrrole providing a N-((3-ethynyl-1-methyl-1H-pyrrol-2-yl)methyl)-N-methylbutan-1-amine labeled peptide.

In certain embodiments, the method further comprises contacting the N-((3-ethynyl-1-methyl-1H-pyrrol-2-yl)methyl)-N-methylbutan-1-amine labeled peptide with a solid surface conjugated to a triazene under conditions such that a N-methyl-N-((1-methyl-3-(1,2,3-triazol-4-yl)-1H-pyrrol-2-yl)methyl) butan-1-amine peptide is conjugated to the solid surface.

In certain embodiments, the label is a ligand providing a N-((1-methyl-1H-pyrrol-2-yl)methyl)-N-methylbutan-1-amine ligand labeled peptide. In certain embodiments, the method further comprises contacting the N-((1-methyl-1H-pyrrol-2-yl)methyl)-N-methylbutan-1-amine ligand labeled peptide with a solid surface conjugated to a receptor under conditions such that N-((1-methyl-1H-pyrrol-2-yl)methyl)-N-methylbutan-1-amine ligand labeled peptide is conjugated to the solid surface.

In certain embodiments, the label is biotin providing a N-((1-methyl-1H-pyrrol-2-yl)methyl)-N-methylbutan-1-amine biotin labeled peptide. In certain embodiments, the method further comprises contacting the N-((1-methyl-1H-pyrrol-2-yl)methyl)-N-methylbutan-1-amine biotin labeled peptide with a solid surface conjugated to avidin or streptavidin under conditions such that N-((1-methyl-1H-pyrrol-2-yl)methyl)-N-methylbutan-1-amine biotin labeled peptide is conjugated to the solid surface.

In certain embodiments, this disclosure relates to methods of modifying a peptide comprising N,N′-dimethyl lysine comprising contacting a peptide having a N,N′-dimethyl lysine amino acid with a cyanide salt and an oxidizing agent under conditions such that 2-(methyl(butyl)amino)acetonitrile is formed in place of N,N′-dimethyl lysine. In certain embodiments, the oxidizing agent is 1-chloromethyl-4-fluoro-1,4-diazoniabicyclo[2.2.2]octane bis(tetrafluoroborate) (Selectfluor™). In certain embodiments, the method further comprises contacting the 2-(methyl(butyl)amino)acetonitrile with labeled furan providing a N-((furan-2-yl)methyl)-N-methylbutan-1-amine labeled peptide.

In certain embodiments, the method further comprises contacting the peptide having a 2-(methylamino)acetonitrile group with 3-ethynylfuran providing a N-((3-ethynylfuran-2-yl)methyl)-N-methylbutan-1-amine labeled peptide.

In certain embodiments, the method further comprises contacting the N-((3-ethynylfuran-2-yl)methyl)-N-methylbutan-1-amine labeled peptide with a solid surface conjugated to a triazene under conditions such that a N-((3-(1,2,3-triazol-4-yl)-furan-2-yl)methyl)-N-methylbutan-1-amine peptide is conjugated to the solid surface.

In certain embodiments, the label is a ligand providing a N-((furan-2-yl)methyl)-N-methylbutan-1-amine ligand labeled peptide. In certain embodiments, the method further comprises contacting the N-((furan-2-yl)methyl)-N-methylbutan-1-amine ligand labeled peptide with a solid surface conjugated to a receptor under conditions such that N-((furan-2-yl)methyl)-N-methylbutan-1-amine ligand labeled peptide is conjugated to the solid surface.

In certain embodiments, the label is biotin providing a N-((furan-2-yl)methyl)-N-methylbutan-1-amine biotin labeled peptide. In certain embodiments, the method further comprises contacting the N-((furan-2-yl)methyl)-N-methylbutan-1-amine biotin labeled peptide with a solid surface conjugated to avidin or streptavidin under conditions such that N-((furan-2-yl)methyl)-N-methylbutan-1-amine biotin labeled peptide is conjugated to the solid surface.

In certain embodiments, this disclosure relates to methods of modifying a peptide comprising N,N′-dimethyl lysine comprising contacting a peptide having a N,N′-dimethyl lysine amino acid with a cyanide salt and an oxidizing agent under conditions such that 2-(methyl(butyl)amino)acetonitrile is formed in place of N,N′-dimethyl lysine. In certain embodiments, the oxidizing agent is 1-chloromethyl-4-fluoro-1,4-diazoniabicyclo[2.2.2]octane bis(tetrafluoroborate) (Selectfluor™). In certain embodiments, the method further comprises contacting the 2-(methyl(butyl)amino)acetonitrile with labeled propan-2-one providing a 4-(butyl(methyl)amino) butan-2-one labeled peptide.

In certain embodiments, the method further comprises contacting the peptide having a 2-(methylamino)acetonitrile group with pent-4-yn-2-one providing a 1-(butyl(methyl)amino) hex-5-yn-3-one labeled peptide.

In certain embodiments, the method further comprises contacting the 1-(butyl(methyl)amino) hex-5-yn-3-one labeled peptide with a solid surface conjugated to a triazene under conditions such that a 4-(butyl(methyl)amino)-1-(1,2,3-triazol-4-yl) butan-2-one peptide is conjugated to the solid surface.

In certain embodiments, the label is a ligand providing a 4-(butyl(methyl)amino) butan-2-one ligand labeled peptide. In certain embodiments, the method further comprises contacting the 4-(butyl(methyl)amino) butan-2-one ligand labeled peptide with a solid surface conjugated to a receptor under conditions such that a 4-(butyl(methyl)amino) butan-2-one ligand labeled peptide is conjugated to the solid surface.

In certain embodiments, the label is biotin providing a 4-(butyl(methyl)amino) butan-2-one biotin labeled peptide. In certain embodiments, the method further comprises contacting the 4-(butyl(methyl)amino) butan-2-one biotin labeled peptide with a solid surface conjugated to avidin or streptavidin under conditions such that a 4-(butyl(methyl)amino) butan-2-one biotin labeled peptide is conjugated to the solid surface.

In certain embodiments, this disclosure relates to methods of modifying a peptide comprising N,N′-dimethyl lysine comprising contacting a peptide having a N,N′-dimethyl lysine amino acid with a cyanide salt and an oxidizing agent under conditions such that 2-(methyl(butyl)amino)acetonitrile is formed in place of N,N′-dimethyl lysine. In certain embodiments, the oxidizing agent is 1-chloromethyl-4-fluoro-1,4-diazoniabicyclo[2.2.2]octane bis(tetrafluoroborate) (Selectfluor™). In certain embodiments, the method further comprises contacting the 2-(methyl(butyl)amino)acetonitrile with allyltrimethylsilane providing a peptide having an N-methyl-N-methylbut-3-en-1-amine group.

In certain embodiments, the method further comprises contacting the peptide having the N-methyl-N-methylbut-3-en-1-amine group with ozone providing a peptide having a 3-(butyl(methyl)amino)propanal group.

In certain embodiments, the method further comprises contacting the peptide having a 3-(butyl(methyl)amino)propanal group with a solid surface comprising a primary amine group under conditions such that a N-(3-(azaneylidene)propyl)-N-methylbutan-1-amine peptide is conjugated to the solid surface.

In certain embodiments, this disclosure relates to methods of modifying a tertiary amine comprising contacting a compound having tertiary amine with a N,N′-dimethyl amine in the tertiary amine with labeled indole and an oxidizing agent under conditions such that a N-((1H-indol-3-yl)methyl)-N-methylamine labeled compound is formed in place of dimethyl amine. In certain the agent embodiments, oxidizing is 1-chloromethyl-4-fluoro-1,4-diazoniabicyclo[2.2.2]octane bis(tetrafluoroborate) (Selectfluor™). In certain embodiments, the label is an alkynyl group providing N-((1H-indol-3-yl)methyl)-N-methylamine alkynyl labeled compound. In certain embodiments, method further comprises contacting the N-((1H-indol-3-yl)methyl)-N-methylamine alkynyl labeled compound with a solid surface conjugated to a triazene under conditions such that N-((1H-indol-3-yl)methyl)-N-methylamine triazole compound is conjugated the solid surface; washing the solid surface providing a purified N-((1H-indol-3-yl)methyl)-N-methylamine triazole compound conjugated to a solid surface; and contacting the purified N-((1H-indol-3-yl)methyl)-N-methylamine triazole compound conjugated to solid surface with an acid solution such that the purified compound is cleaved from the solid surface providing purified compound.

In certain embodiments, the label is a ligand providing a N-((1H-indol-3-yl)methyl)-N-methylamine ligand labeled compound. In certain embodiments, the method further comprises contacting the N-((1H-indol-3-yl)methyl)-N-methylamine ligand labeled compound with a solid surface conjugated to a receptor of the ligand under conditions such that a N-((1H-indol-3-yl)methyl)-N-methylamine conjugated compound is conjugated to the solid surface; and washing the solid surface providing a purified conjugated compound on the solid surface.

In certain embodiments, the label is biotin providing a N-((1H-indol-3-yl)methyl)-N-methylamine biotin labeled compound. In certain embodiments, the method further comprises contacting the N-((1H-indol-3-yl)methyl)-N-methylamine biotin labeled compound with a solid surface conjugated to avidin or streptavidin under conditions such that N-((1H-indol-3-yl)methyl)-N-methylamine biotin conjugated compound is conjugated to the solid surface; and washing the solid surface providing a purified compound conjugated to the solid surface.

In certain embodiments, this disclosure relates to methods of modifying a peptide comprising a N,N′-dimethyl lysine comprising contacting a peptide with a N,N′-dimethyl lysine amino acid with labeled indole and an oxidizing agent under conditions such that N-((1H-indol-3-yl)methyl)-N-methylbutan-1-amine labeled peptide is formed in place of N,N′-dimethyl lysine. In certain embodiments, the oxidizing is 1-chloromethyl-4-fluoro-1,4-agent diazoniabicyclo[2.2.2]octane bis(tetrafluoroborate) (Selectfluor™). In certain embodiments, the label is an alkynyl group providing N-((1H-indol-3-yl)methyl)-N-methylbutan-1-amine alkynyl labeled peptide. In certain embodiments, the method further comprises contacting the N-((1H-indol-3-yl)methyl)-N-methylbutan-1-amine alkynyl labeled peptide with a solid surface conjugated to a triazene under conditions such that N-((1H-indol-3-yl)methyl)-N-methylbutan-1-amine triazole peptide is conjugated to the solid surface; washing the solid surface providing a purified N-((1H-indol-3-yl)methyl)-N-methylbutan-1-amine triazole peptide conjugated to the solid surface; and contacting the purified N-((1H-indol-3-yl)methyl)-N-methylbutan-1-amine triazole peptide conjugated to the solid surface with an acid solution such that the purified peptide is cleaved from the solid surface providing purified peptide.

In certain embodiments, the label is ligand providing N-((1H-indol-3-yl)methyl)-N-methylbutan-1-amine ligand labeled peptide. In certain embodiments, the method further comprises contacting the N-((1H-indol-3-yl)methyl)-N-methylbutan-1-amine ligand labeled peptide with a solid surface conjugated to a receptor of the ligand under conditions such that N-((1H-indol-3-yl)methyl)-N-methylbutan-1-amine biotin conjugated peptide is conjugated to the solid surface; and washing the solid surface providing a purified peptide conjugated to the solid surface.

In certain embodiments, the label is biotin providing an N-((1H-indol-3-yl)methyl)-N-methylbutan-1-amine biotin labeled peptide. In certain embodiments, the method further comprises contacting the N-((1H-indol-3-yl)methyl)-N-methylbutan-1-amine biotin labeled peptide with a solid surface conjugated to avidin or streptavidin under conditions such that N-((1H-indol-3-yl)methyl)-N-methylbutan-1-amine biotin conjugated peptide is conjugated to the solid surface; and washing the solid surface providing a purified peptide conjugated to the solid surface.

In certain embodiments, this disclosure relates to methods of modifying a compound comprising contacting a tertiary amine compound having a N,N′-dimethyl amine group with an alkynyl and an oxidizing agent under conditions such that an N-methyl-N-(alkyn-1-yl)amine labeled compound is formed in place of dimethyl amine. In certain embodiments, the oxidizing agent is 1-chloromethyl-4-fluoro-1,4-diazoniabicyclo[2.2.2]octane bis(tetrafluoroborate) (Selectfluor™). In certain embodiments, the alkynyl is ethynyl phenyl and an N-methyl-N-(3-phenylprop-2-yn-1-yl)amine labeled compound is formed in place of dimethyl amine. In certain embodiments, the method further comprises contacting the N-methyl-N-(3-phenylprop-2-yn-1-yl)amine labeled compound with a solid surface conjugated to triazene under conditions such that N-methyl-N-((5-phenyl-1,2,3-triazol-4-yl)methyl)amine compound is conjugated to the solid surface; and washing the solid surface providing a purified N-methyl-N-((5-phenyl-1,2,3-triazol-4-yl)methyl)amine compound conjugated to the solid surface.

In certain embodiments, this disclosure relates to methods of modifying a peptide comprising N,N′-dimethyl lysine comprising contacting a peptide with a N,N′-dimethyl lysine amino acid with an alkynyl and an oxidizing agent under conditions such that an N-methyl-N-(alkyn-1-yl) butan-1-amine labeled peptide is formed in place of N,N′-dimethyl lysine. In certain embodiments, the oxidizing agent is 1-chloromethyl-4-fluoro-1,4-diazoniabicyclo[2.2.2]octane bis(tetrafluoroborate) (Selectfluor™). In certain embodiments, the alkynyl is ethynyl phenyl and an N-methyl-N-(3-phenylprop-2-yn-1-yl) butan-1-amine labeled peptide is formed in place of N,N′-dimethyl lysine. In certain embodiments, the method further comprises contacting the N-methyl-N-(3-phenylprop-2-yn-1-yl) butan-1-amine labeled peptide with a solid surface conjugated to triazene under conditions such that N-methyl-N-((5-phenyl-1,2,3-triazol-4-yl)methyl) butan-1-amine peptide is conjugated to the solid surface; and washing the solid surface providing a purified N-methyl-N-((5-phenyl-1,2,3-triazol-4-yl)methyl) butan-1-amine peptide conjugated to the solid surface.

In certain embodiments, this disclosure relates to methods of modifying a compound comprising contacting a tertiary amine compound having a N,N′-dimethyl amine group with 4-dimethylamino pyridine, pyridine, or other pyridine having a para substituted tertiary amine, and an oxidizing agent under conditions such that an aldehyde group is formed in place of N,N′-dimethyl amine, e.g., a converting dimethyl lysine side chain into butyraldehyde. In certain embodiments, the oxidizing agent is 1-chloromethyl-4-fluoro-1,4-diazoniabicyclo[2.2.2]octane bis(tetrafluoroborate) (Selectfluor™). In certain embodiments, the compound is a peptide or protein having a N,N′-dimethyl lysine amino acid.

In certain embodiments, the method further comprises contacting the compound having the aldehyde group with primary amine group, alkyl amine, aniline, or hydrazine group providing a compound having a hydrazine or imine Schiff base linkage, e.g., 1-butylidene-2-methylhydrazine compound or N-methylbutan-1-imine compound optionally labeled. In certain embodiments, the label is an alkynyl group, biotin, or fluorinated hydrocarbon.

In certain embodiments, the method further comprises contacting the compound having aldehyde group with a solid surface comprising primary amine group, alkyl amine, aniline, or hydrazine group under conditions such that a compound having a hydrazine or imine Schiff base linkage, e.g., 1-butylidene-2-methylhydrazine compound or N-methylbutan-1-imine compound is conjugated to the solid surface.

In certain embodiments, the method further comprises contacting the compound having aldehyde group with a solid surface comprising a primary amine group, alkyl amine, aniline, or hydrazine group under conditions such that a labeled compound having a hydrazine or imine Schiff base linkage. In certain embodiments, the labeled compound is an alkynyl labeled compound providing 1-butylidene-2-(prop-2-yn-1-yl) hydrazine compound.

In certain embodiments, the method further comprises contacting the alkynyl labeled compound with a triazene providing a triazole labeled compound. In certain embodiments, the triazene is conjugated to a solid surface providing a triazole linkage to the compound or peptide.

In certain embodiments, the label is a ligand providing a 1-butylidene-2-methylhydrazine ligand labeled peptide. In certain embodiments, the method further comprises contacting the 1-butylidene-2-methylhydrazine ligand labeled peptide with a solid surface conjugated to a receptor under conditions such that the 1-butylidene-2-methylhydrazine ligand labeled peptide is conjugated to the solid surface.

In certain embodiments, the label is biotin providing a 1-butylidene-2-methylhydrazine biotin labeled peptide. In certain embodiments, the method further comprises contacting the 1-butylidene-2-methylhydrazine biotin labeled peptide with a solid surface conjugated to avidin or streptavidin under conditions such that the 1-butylidene-2-methylhydrazinebiotin labeled peptide is conjugated to the solid surface.

In certain embodiments, this disclosure relates to methods of modifying a peptide comprising contacting a compound or peptide having an N-terminal N,N′-dimethyl amine group with a cyanide salt and an oxidizing agent under conditions such that a peptide substituted with a nitrile group is formed; and contacting the peptide substituted with a nitrile group with a labeled 2-aminoethane-1-thiol providing a thiazolidine labeled compound. In certain embodiments, the oxidizing agent is 1-chloromethyl-4-fluoro-1,4-diazoniabicyclo[2.2.2]octane bis(tetrafluoroborate) (Selectfluor).

In certain embodiments, the label is an alkynyl group providing alkynyl labeled peptide. In certain embodiments, methods further comprise contacting the alkynyl labeled peptide with a solid surface conjugated to a triazene under conditions such that a triazole peptide is conjugated to the solid surface.

In certain embodiments, methods further comprise contacting the triazole peptide conjugated to the solid surface with an acid solution such that the purified peptide is cleaved from the solid surface providing purified peptide.

In certain embodiments, this disclosure relates to methods of modifying a tertiary amine comprising contacting a compound or peptide having an 2-(dimethylamino)acetamide group optionally substituted on the alpha carbon with an N-terminal N,N′-dimethyl amine with labeled 2-aminoethane-1-thiol and an oxidizing agent under conditions such that a thiazolidine labeled compound is formed in place of dimethyl amine and the alpha nitrile forming a labeled thiazolidine-2-carboxamide ring connected at the carbon between the nitrogen and sulfur atoms of the thiazolidine ring. In certain embodiment, the labeled thiazolidine-2-carboxamide ring connected at the carbon between the nitrogen and sulfur atoms of the thiazolidine ring to the side chain carbon of the N-terminal amino acid is a peptide. In certain embodiments, the oxidizing agent is 1-chloromethyl-4-fluoro-1,4-diazoniabicyclo[2.2.2]octane bis(tetrafluoroborate) (Selectfluor™).

In certain embodiments, the label is an alkynyl group providing a thiazolidine alkynyl labeled compound. In certain embodiments, the method further comprises contacting the thiazolidine alkynyl labeled compound with a solid surface conjugated to a triazene under conditions such that a thiazolidine triazole compound is conjugated the solid surface; washing the solid surface providing a purified thiazolidine triazole compound conjugated to a solid surface; and contacting the purified thiazolidine triazole compound conjugated to solid surface with an acid solution such that the purified compound or peptide is cleaved from the solid surface providing purified compound or peptide.

In certain embodiments, the label is a ligand providing a thiazolidine ligand labeled compound. In certain embodiments, the method further comprises contacting the thiazolidine ligand labeled compound with a solid surface conjugated to a receptor of the ligand under conditions such that a thiazolidine conjugated compound is conjugated to the solid surface; and washing the solid surface providing a purified thiazolidine conjugated compound on the solid surface.

In certain embodiments, the label is biotin providing a thiazolidine biotin labeled compound. In certain embodiments, the method further comprises contacting the thiazolidine biotin labeled compound with a solid surface conjugated to avidin or streptavidin under conditions such that the thiazolidine biotin conjugated compound is conjugated to the solid surface; and washing the solid surface providing a purified thiazolidine compound conjugated to the solid surface.

In certain embodiments, this disclosure relates to methods of modifying a peptide comprising an N-terminal N,N′-dimethyl group comprising contacting a peptide or compound with an N-terminal 2-(dimethylamino)acetamide with labeled 2-aminoethane-1-thiol and an oxidizing agent under conditions such that a thiazolidine labeled peptide or compound is formed in place of dimethyl amine and the alpha nitrile forming a labeled thiazolidine-2-carboxamide ring connected at the carbon between the nitrogen and sulfur atoms of the thiazolidine ring. In certain embodiments, the oxidizing agent is 1-chloromethyl-4-fluoro-1,4-diazoniabicyclo[2.2.2]octane bis(tetrafluoroborate) (Selectfluor™).

In certain embodiments, the label is an alkynyl group providing a thiazolidine alkynyl labeled peptide. In certain embodiments, the method further comprises contacting the thiazolidine alkynyl labeled peptide with a solid surface conjugated to a triazene under conditions such that a thiazolidine triazole peptide is conjugated to the solid surface; washing the solid surface providing a purified thiazolidine triazole peptide conjugated to the solid surface; and contacting the thiazolidine triazole peptide conjugated to the solid surface with an acid solution such that the purified peptide is cleaved from the solid surface providing purified peptide.

In certain embodiments, this disclosure relates to methods of modifying a peptide or compound comprising a N,N′-dimethyl amino group comprising contacting a peptide or compound with a N,N′-dimethyl amino group with a labeling reagent and an oxidizing agent under conditions such that a labeled peptide is formed. In certain embodiments, the oxidizing agent is 1-chloromethyl-4-fluoro-1,4-diazoniabicyclo[2.2.2]octane bis(tetrafluoroborate) (Selectfluor™). In certain embodiments, the method further comprises contacting the labeled peptide with a solid surface conjugated to a receptor or other labeled reactive group, e.g., triazene, under conditions such that the compound or peptide is conjugated to the solid surface; washing the solid surface providing a purified compound or peptide conjugated to the solid surface; and contacting the purified compound or peptide conjugated to the solid surface with an removal solution, e.g. acid solution such that the purified compound or peptide is cleaved from the solid surface providing a purified providing peptide or compound.

In certain embodiments, for any of the methods disclosed herein the label may be biotin, an aromatic molecule, a fluorescent dye, a second alkynyl group, a ligand, a receptor, an antibody, an antigen, or fluorinated hydrocarbon.

In certain embodiments, for any of the methods disclosed herein the methods further comprise determining the molecular weight of the peptide or compound.

In certain embodiments, for any of the methods disclosed herein the methods further comprise separating a purified, cleaved, or isolated peptide composition or compound composition into two or more peptides or compounds. In certain embodiments, separating is by chromatography.

In certain embodiments, for any of the methods disclosed herein the triazole compound conjugated to solid surface is contacted with an acid solution such that a purified compound is cleaved from the solid surface providing purified compound.

In certain embodiments, for any of the methods disclosed herein the washing step is optionally omitted.

In certain embodiments, for any of the methods disclosed herein reactions are in an aqueous solution with a pH of between 6.5 to 8.5. In certain embodiments, for any of the methods disclosed herein reactions are in an aqueous solution with a pH of between 7.0 to 8.5. In certain embodiments, for any of the methods disclosed herein reactions are in an aqueous solution with a pH of between 7.0 to 8.0.

In certain embodiments, for any of the methods disclosed herein reactions are at about room temperature, e.g., between 10 and 30 degrees Celsius. In certain embodiments, for any of the methods disclosed herein reactions are at between 5 and 40 degrees Celsius.

In certain embodiments, for any of the methods disclosed herein methods of contacting a tertiary amine or compound or peptide containing a tertiary amine or dimethyl lysine wherein the tertiary amine or compound or peptide containing a tertiary amine or dimethyl lysine is in a sample such as a biological sample, (e.g., cell, tissue, etc.) or environmental sample. Biological samples may be obtained from animals (including humans) and encompass fluids, blood, solids, tissues, and gases. Environmental samples include environmental material such as surface matter, soil, water, and industrial samples.

### Compositions

In certain embodiments, this disclosure relates to compositions, compounds, and peptides having the following formula:

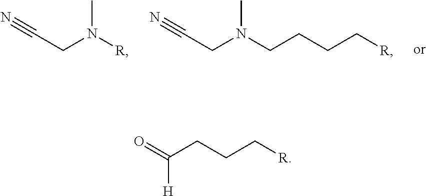

or salts or derivative thereof wherein,
R is a peptide, protein, or compound wherein the peptide, protein, or compound is optionally substituted or conjugated to a label or a solid support through a linking group.

In certain embodiments, the compound is peptide, protein, amino acid, nucleotide, nucleic acid, DNA, RNA, nucleobase polymer, alkyl, aryl, carbocyclyl, heteroaryl optionally substituted with one or more, the same or different substituents such as a halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, which is optionally further substituted or conjugated to a label or a solid support through a linking group.

In certain embodiments, this disclosure relates to peptides, proteins, or compounds having the following formula:

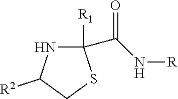

or salts or derivative thereof wherein,
R is a peptide, protein, or compound wherein the peptide, protein, or compound is optionally substituted, conjugated to a label or a solid support through a linking group;
R¹ is hydrogen or an amino acid side chain or alkyl group optionally substituted with R¹⁰;
R² is a label, hydrogen, alkyl, formyl, alkanoyl, carboxyl, carbamoyl, aryl, carbocyclyl or heterocyclyl, or N-substituted carbamoyl group R² is optionally substituted with R¹⁰, conjugated to a label, or a solid support through a linking group;
R¹⁰ is halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, wherein R¹⁰ is optionally substituted, conjugated to a label, or a solid support through a linking group.

In certain embodiments, R² is conjugated to a label, or a solid support through a linking group.

In certain embodiments, the compound is peptide, protein, amino acid, nucleotide, nucleic acid, DNA, RNA, nucleobase polymer, alkyl, aryl, carbocyclyl, heteroaryl optionally substituted with one or more, the same or different substituents such as a halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, which is optionally further substituted or conjugated to a label or a solid support through a linking group.

In certain embodiments, this disclosure relates to peptides, proteins, or compounds having the following formula:

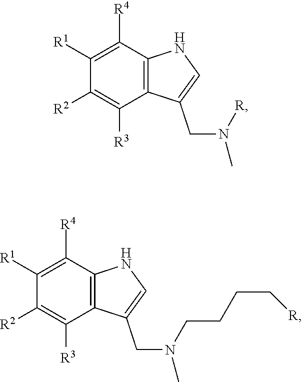

or salts or derivative thereof wherein,
R is a peptide, protein, or compound wherein the peptide, protein, or compound is optionally substituted, conjugated to a label or a solid support through a linking group;
R¹, R², R³, and R⁴ are individually and independently at each occurrence hydrogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group R¹, R², R³, and R⁴ are optionally substituted with R¹⁰, conjugated to a label, or a solid support through a linking group; or two of R¹, R², R³, and R⁴ come together with the attached atoms to for an aromatic or non-aromatic ring optionally substituted with R⁶, and
R¹⁰ is halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, wherein R¹⁰ is optionally substituted, conjugated to a label, or a solid support through a linking group.

In certain embodiments, R¹ is conjugated to a label, or a solid support through a linking group.

In certain embodiments, the compound is peptide, protein, amino acid, nucleotide, nucleic acid, DNA, RNA, nucleobase polymer, alkyl, aryl, carbocyclyl, heteroaryl optionally substituted with one or more, the same or different substituents such as a halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, which is optionally further substituted or conjugated to a label or a solid support through a linking group.

In certain embodiments, this disclosure relates to peptides, proteins, or compounds having the following formula:

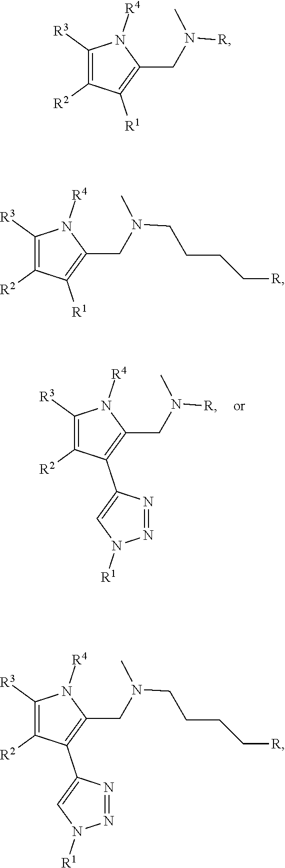

or salts or derivative thereof wherein,
R is a peptide, protein, or compound wherein the peptide, protein, or compound is optionally substituted, conjugated to a label or a solid support through a linking group;
R¹, R², R³, and R⁴ are individually and independently at each occurrence hydrogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group wherein R¹, R², R³, and R⁴ are optionally substituted with R¹⁰, conjugated to a label, or a solid support through a linking group; or two or R¹, R², R³, and R⁴ are individually and independently at each occurrence come together with the attached atoms to for an aromatic or non-aromatic ring optionally substituted with R¹⁰, and
R¹⁰ is halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, wherein R¹⁰ is optionally substituted, conjugated to a label, or a solid support through a linking group.

In certain embodiments, R¹ is conjugated to a label, or a solid support through a linking group.

In certain embodiments, the compound is peptide, protein, amino acid, nucleotide, nucleic acid, DNA, RNA, nucleobase polymer, alkyl, aryl, carbocyclyl, heteroaryl optionally substituted with one or more, the same or different substituents such as a halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, which is optionally further substituted or conjugated to a label or a solid support through a linking group.

In certain embodiments, this disclosure relates to peptides, proteins, or compounds having the following formula:

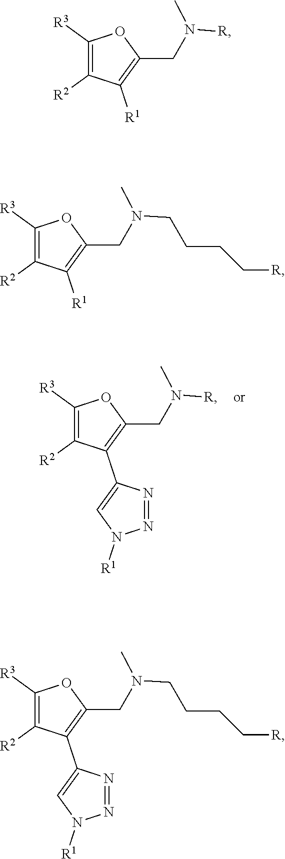

or salts or derivative thereof wherein,
R is a peptide, protein, or compound wherein the peptide, protein, or compound is optionally substituted, conjugated to a label or a solid support through a linking group;
R¹, R², and R³ are individually and independently at each occurrence hydrogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group wherein R¹, R², and R³ optionally substituted with R¹⁰, conjugated to a label, or a solid support through a linking group; or two or R¹, R², and R³ come together with the attached atoms to for an aromatic or non-aromatic ring optionally substituted with R¹⁰, and
R¹⁰ is halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, wherein R⁶ is optionally substituted, conjugated to a label, or a solid support through a linking group.

In certain embodiments, R¹ is conjugated to a label, or a solid support through a linking group.

In certain embodiments, the compound is peptide, protein, amino acid, nucleotide, nucleic acid, DNA, RNA, nucleobase polymer, alkyl, aryl, carbocyclyl, heteroaryl optionally substituted with one or more, the same or different substituents such as a halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, which is optionally further substituted or conjugated to a label or a solid support through a linking group.

In certain embodiments, this disclosure relates to peptides, proteins, or compounds having the following formula:

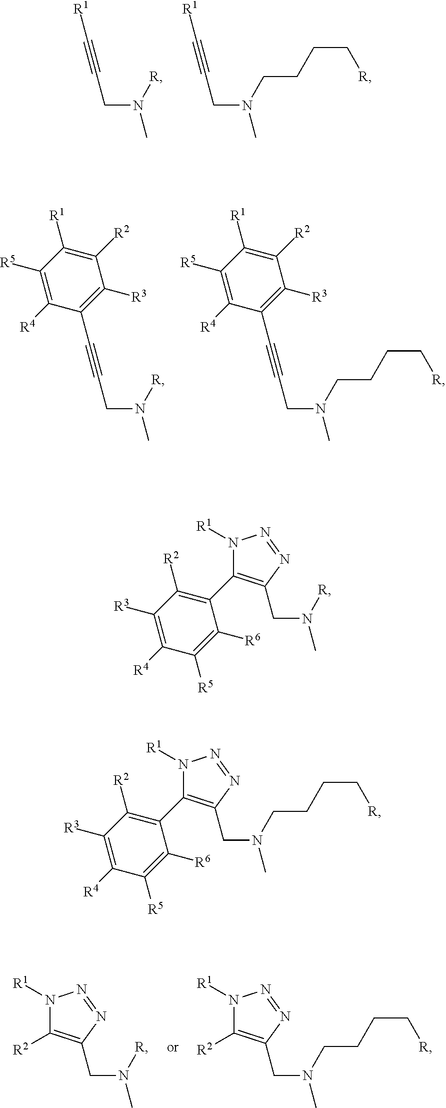

or salts or derivative thereof wherein,
R is a peptide, protein, or compound wherein the peptide, protein, or compound is optionally substituted, conjugated to a label or a solid support through a linking group;
R¹, R², R³, R⁴, R⁵ and R⁶ are individually and independently at each occurrence hydrogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group wherein R¹, R², R³, R⁴, R⁵ and R⁶ are optionally substituted with R¹⁰, conjugated to a label, or a solid support through a linking group; or two or R¹, R², R³, R⁴, R⁵ and R⁶ come together with the attached atoms to for an aromatic or non-aromatic ring optionally substituted with R¹⁰, and
R¹⁰ is halogen, hydroxy, amino, thiol, alkyl, alkoxy, alkylamino, alkylthiol, dialkylamino, acetamido, formyl, alkanoyl, carboxyl, carbamoyl, aryl, carbocyclyl or heterocyclyl, or N-substituted carbamoyl group, wherein R² is optionally substituted, conjugated to a label, or a solid support through a linking group.

In certain embodiments, R¹ is conjugated to a label, or a solid support through a linking group.

In certain embodiments, the compound is peptide, amino acid, nucleotide, nucleic acid, DNA, RNA, nucleobase polymer, alkyl, aryl, carbocyclyl, heteroaryl optionally substituted with one or more, the same or different substituents such as a halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, which is optionally further substituted or conjugated to a label or a solid support through a linking group.

In certain embodiments, this disclosure relates to peptides, proteins, or compounds having the following formula:

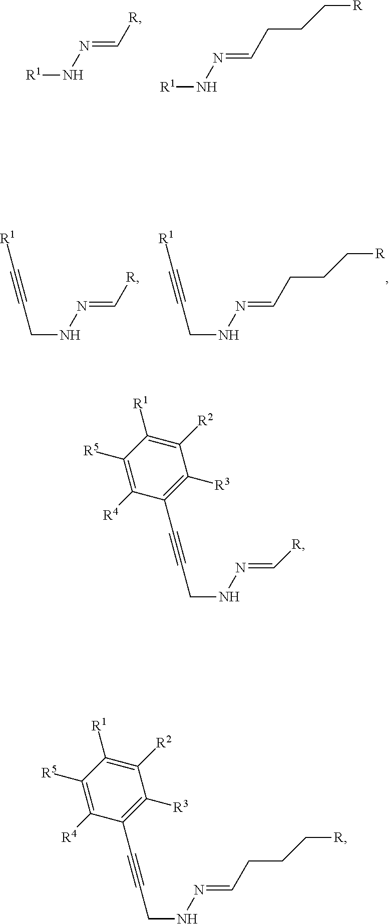

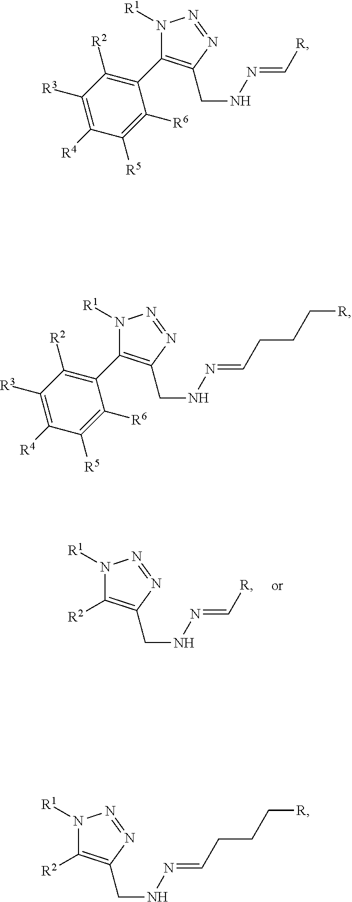

or salts or derivative thereof wherein,
R is a peptide, protein, or compound wherein the peptide, protein, or compound is optionally substituted, conjugated to a label or a solid support through a linking group;
R¹, R², R³, R⁴, R⁵ and R⁶ are individually and independently at each occurrence hydrogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group wherein R¹, R², R³, R⁴, R⁵ and R⁶ are optionally substituted with R¹⁰, conjugated to a label, or a solid support through a linking group; or two or R¹, R², R³, R⁴, R⁵ and R⁶ come together with the attached atoms to for an aromatic or non-aromatic ring optionally substituted with R¹⁰, and
R¹⁰ is halogen, hydroxy, amino, thiol, alkyl, alkoxy, alkylamino, alkylthiol, dialkylamino, acetamido, formyl, alkanoyl, carboxyl, carbamoyl, aryl, carbocyclyl or heterocyclyl, or N-substituted carbamoyl group, wherein R² is optionally substituted, conjugated to a label, or a solid support through a linking group.

In certain embodiments, R¹ is conjugated to a label, or a solid support through a linking group.

In certain embodiments, the compound is peptide, amino acid, nucleotide, nucleic acid, DNA, RNA, nucleobase polymer, alkyl, aryl, carbocyclyl, heteroaryl optionally substituted with one or more, the same or different substituents such as a halogen, hydroxy, amino, thiol, alkyl, alkoxy, alkylamino, alkylthio, dialkylamino, acetamido, formyl, alkanoyl, carboxyl, carbamoyl, aryl, carbocyclyl or heterocyclyl, or N-substituted carbamoyl group, which is optionally further substituted or conjugated to a label or a solid support through a linking group.

In certain embodiments, this disclosure relates to a compound or material comprising or coated with or conjugate to chemical arrangements disclosed herein.

With regard to any of the embodiments disclosed herein, a solid support is a particle, magnetic particle, resin, glass, plastic, silicon, wafer, or multi-well plate (polystyrene microtiter plate).

In certain embodiments, a material, solid support, or particle further comprises or is coated with or conjugated to a hydrophilic polymer.

### EXAMPLES

### Tertiary Amine Coupling by Oxidation (TACO)

In order to elucidate the function of lysine dimethylation of peptides, methods were investigated to determine the feasibility of covalently labeling tertiary amines/dimethyl lysine Kme2 in a selective manner without modification of any other amino acids under physiological conditions.

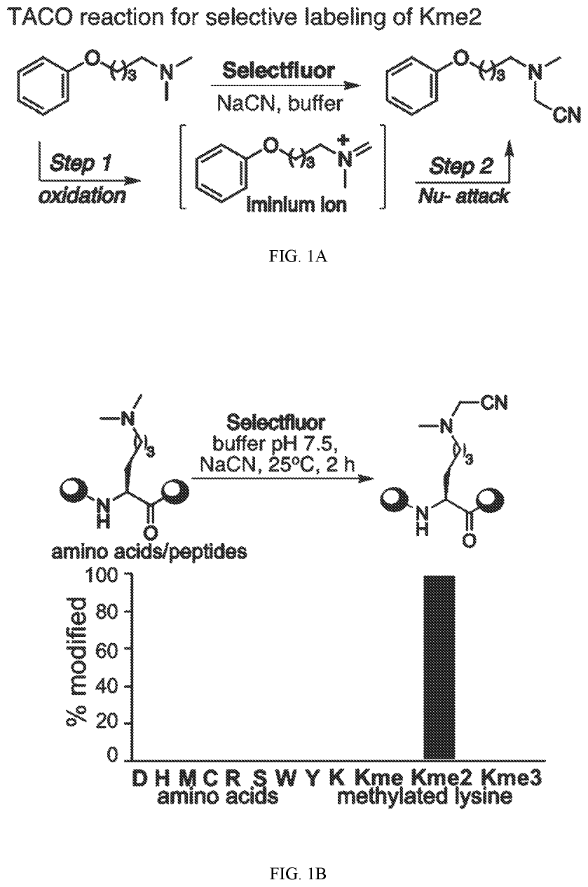

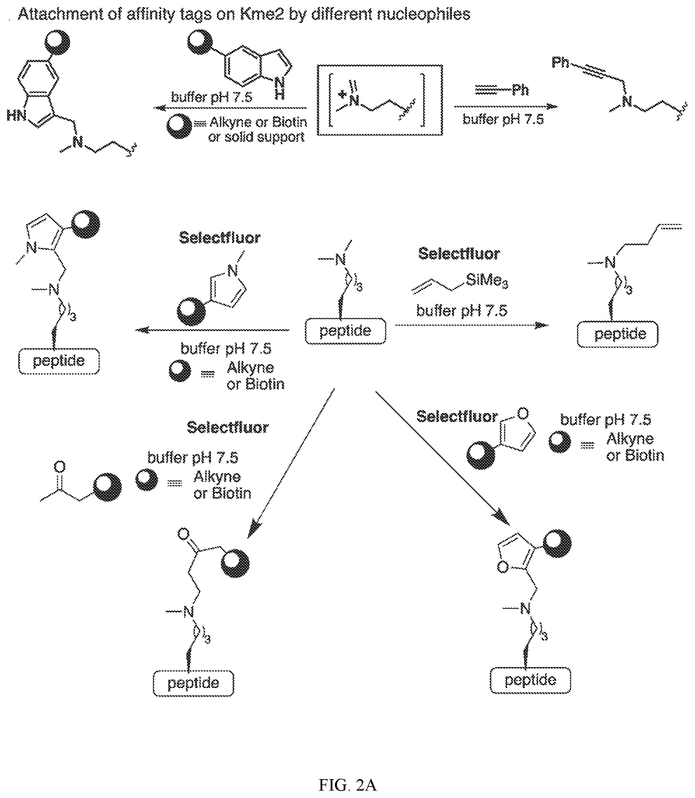

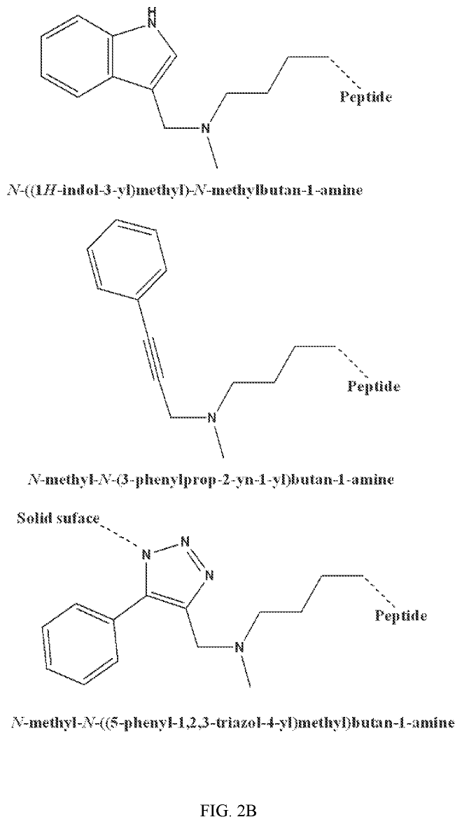

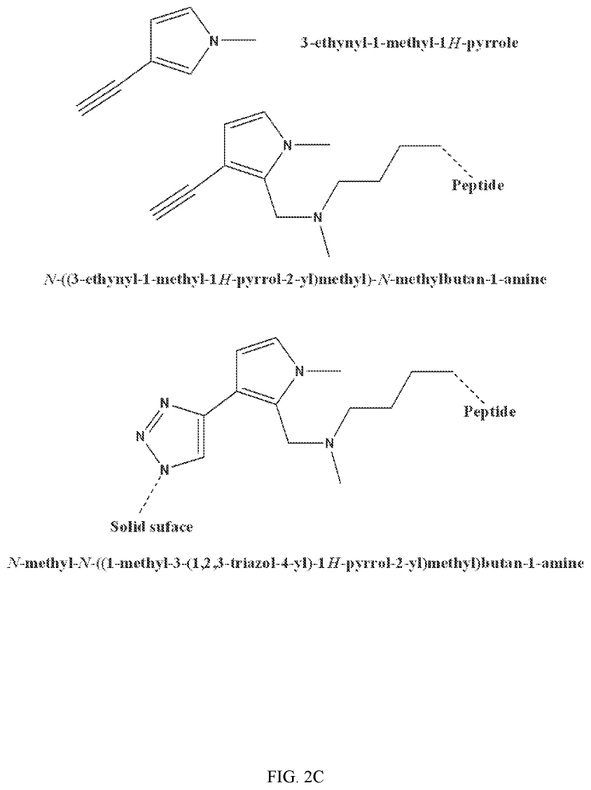

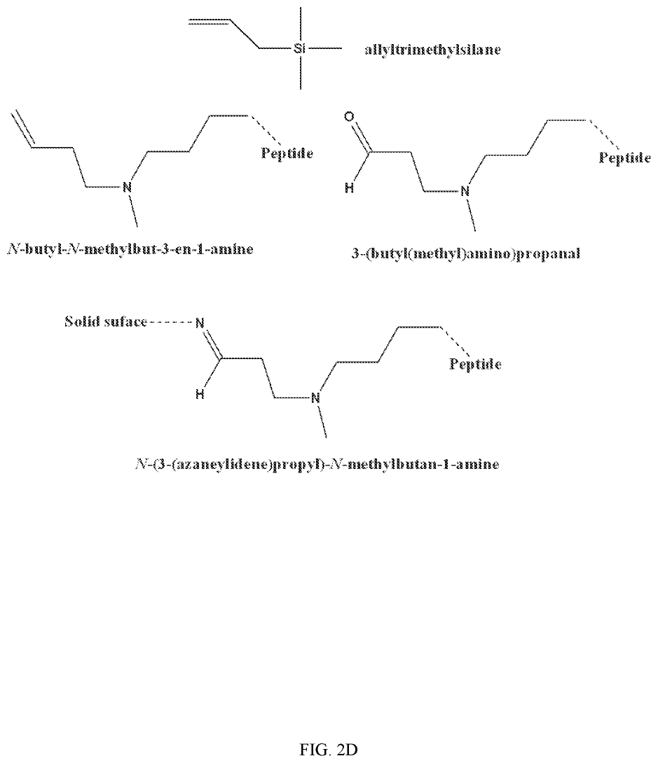

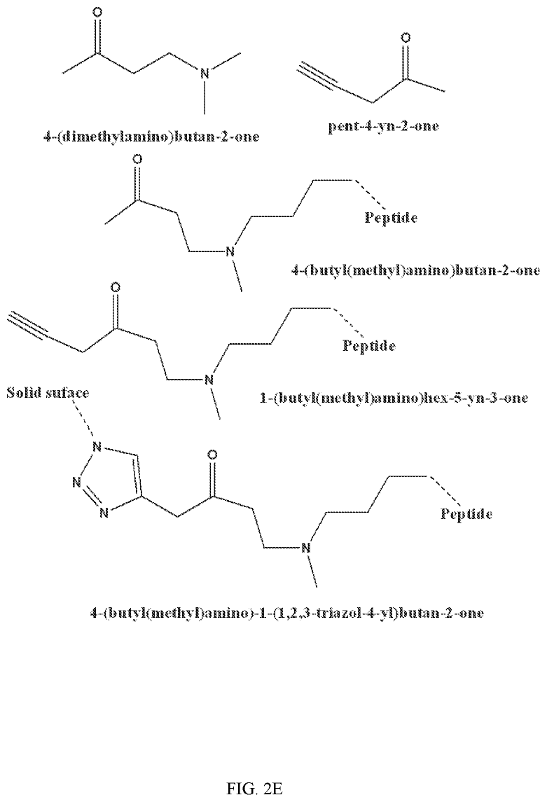

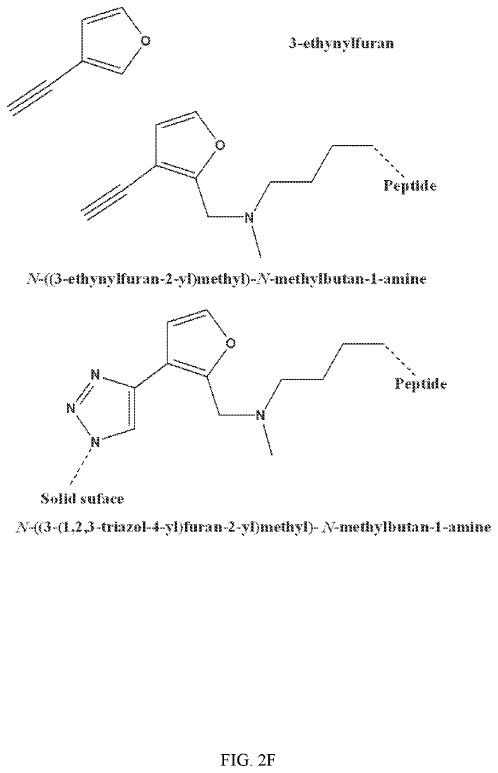

The ability of tertiary amines to form electrophilic iminium ions under oxidative conditions followed by nucleophilic addition was evaluated. It is most desirable if reactions with nucleophiles results in robust labeling of tertiary amines under physiological conditions with the formation of a stable C—C bond between nucleophiles and tertiary amines. A model tertiary amine mimicking the side chain of dimethyl Lysine (Kme2) was used in initial experiments (FIG. 1A). A variety of oxidizing reagents such as CuCl/CuOTf, tBuOOH with FeCl₂, or CoCl₂, tropylium tetrafluoroborate, Selectfluor™ (1-chloromethyl-4-fluoro-1,4-diazoniabicyclo[2.2.2]octane bis(tetrafluoroborate), and various nucleophiles such as NaCN and TMSCN for the modification to a nitrile on the dimethyl group. Of the reactions, the reaction with Selectfluor™ and NaCN resulted in the cyanation of tertiary amines and full conversion to the labeled product was achieved in 1 h under buffer pH 7.5 at room temperature. The cyanation product of a tertiary amine was confirmed by NMR and LCMS.

The chemoselectivity of TACO reaction using Selectfluor™ and NaCN was evaluated with various reactive amino acids such as (D, H, M, C, R, S, W, Y) and varying lysine methylation states (K, Kme, Kme2 and Kme3) (FIG. 1B). The cyanation product was formed with Kme2 without modification of any other amino acids under the reaction conditions. Experiments were performed to determine pan specificity of the TACO method by carrying out reactions with various peptides of different sizes and amino acid compositions with Kme2 at varying positions. A peptide GKme2AKme2F (SEQ ID NO: 1) with alternate Kme2 showed the double cyanation at both Kme2 sites.

Tertiary Amine Coupling by Oxidation (TACO) Based Tertiary Amine (TA) Traps with Affinity Tags.

For effective reaction for the enrichment of Kme2 from complex mixtures, it is desirable to attach affinity tags. It is contemplated that this can be achieved in multiple ways. It is contemplated that one can trap iminium ion generated by oxidation of Kme2 with nucleophiles such as phenylacetylene resulting in the attachment of the alkyne affinity group directly to Kme2 for enrichment.

It is contemplated that one can trap iminium ions by nucleophilic indoles owing to their derivatization with affinity tags such as alkyne or biotin resulting in the attachment of biotin or alkyne group directly to tertiary amines for enrichment by streptavidin beads or azide functionalized beads using click chemistry, respectively.

It is contemplated that one can trap iminium ion generated by oxidation of Kme2 by nucleophiles such as pyrrole resulting in the attachment of affinity tags such as alkyne or biotin directly to Kme2 for enrichment.

It is contemplated that one can trap iminium ion generated by oxidation of Kme2 by nucleophiles such as allyltrimethylsilane resulting in the attachment of the alkene group directly to Kme2 for enrichment.

It is contemplated that one can trap iminium ion generated by oxidation of Kme2 by nucleophiles such as ketone resulting in the attachment of affinity tags such as alkyne or biotin directly to Kme2 for enrichment.

It is contemplated that one can trap iminium ion generated by oxidation of Kme2 by nucleophiles such as furan resulting in the attachment of the affinity tags such as alkyne or biotin directly to Kme2 for enrichment.

### Peptide Aldehyde Formation

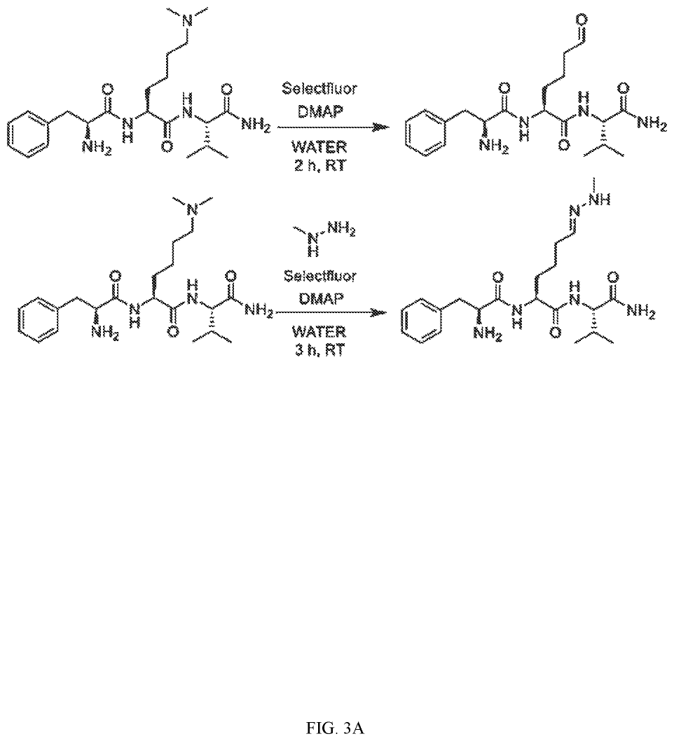

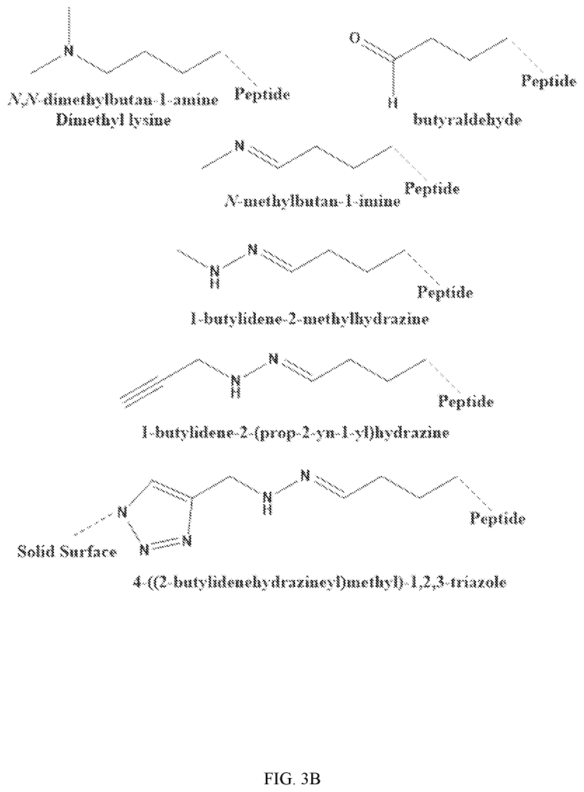

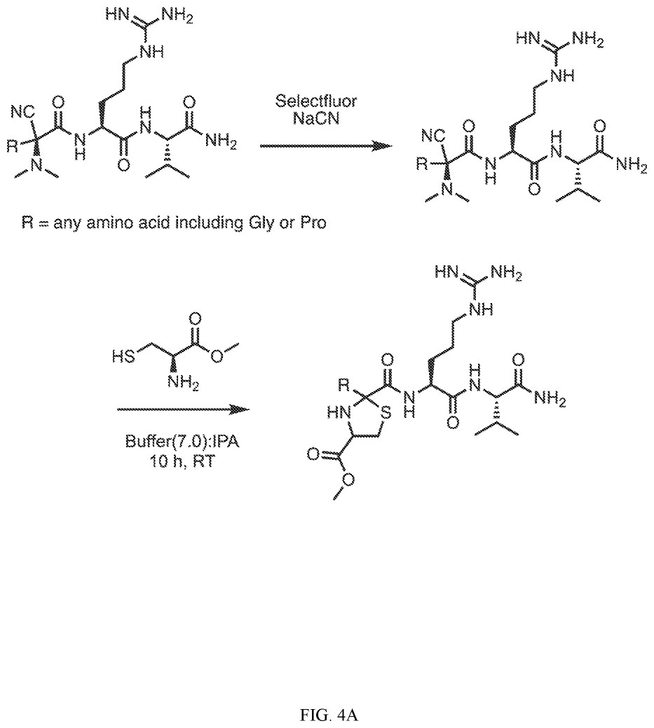

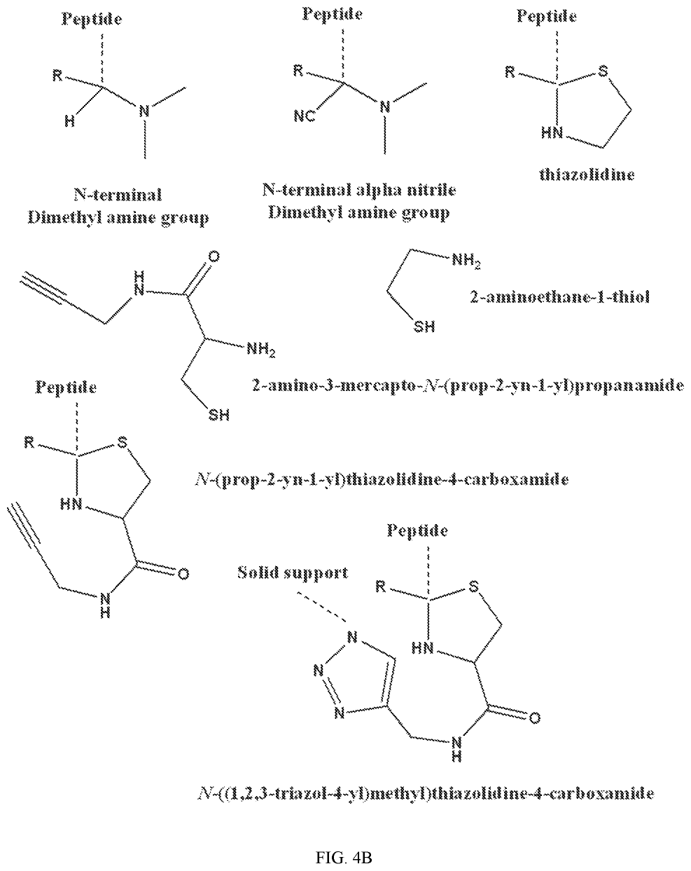

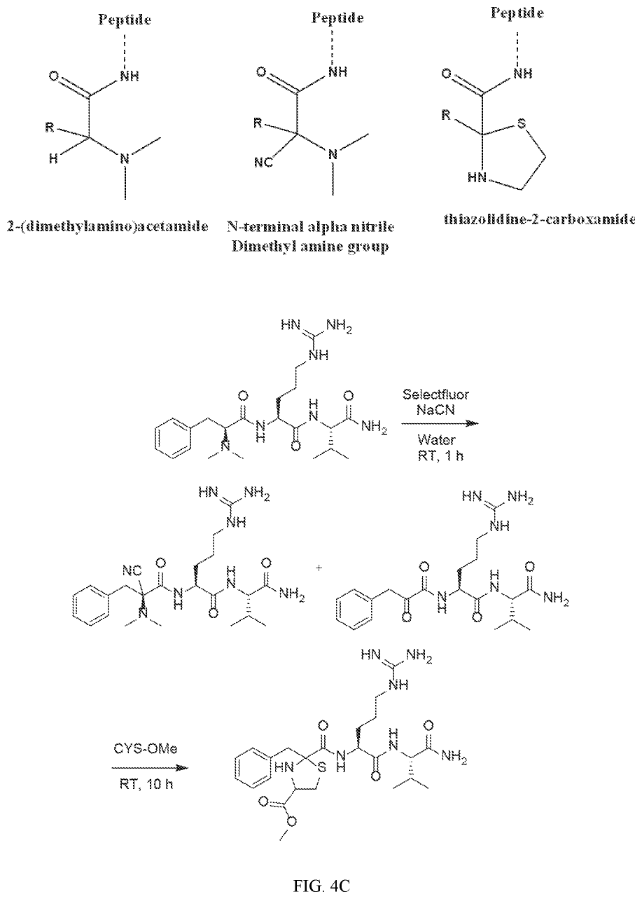

To a stirring solution of dimethyl lysine containing peptide (1 mg, 0.0024 mmol) in water (200 μl), was added successively 4-N,N-dimethylaminopyridine (1.464 mg, 0.012 mmol), and Selectfluor™ (4.248 mg, 0.012 mmol). The reaction mixture was allowed to stir for 2 hours at room temperature. Reaction mixture was purified with IHPC to obtain the peptide aldehyde (See FIG. 3A).

### One-Pot Peptide Aldehyde Formation and Trapping

To a stirring solution of dimethyl lysine containing peptide (1 mg, 0.0024 mmol) in water (200 μL), was added successively 4-N,N-dimethylaminopyridine (1.464 mg, 0.012 mmol), Selectfluor™ (4.248 mg, 0.012 mmol), and hydrazine (0.552 mg, 0.012 mmol). The reaction mixture was allowed to stir for 3 hours at room temperature. Reaction mixture was purified with HPLC to obtain the peptide hydrazone. (FIG. 3B)

Oxidative Cyanation of Tertiary Amines Followed by Trapping with Cysteine for the Selective Covalent Labeling of N-Terminal Dimethyl Groups (NMe2)

Although both NMe2 and Kme2 are tertiary amines, pan-specific method was developed to selectively label N-terminal dimethyl groups (NMe2) on peptide in presence of Kme2 in the same peptide, independent of the sequence and nearby post translational modification (PTMs). Moreover, the selective labeling of NMe2 mostly independent of the amino acid at the N-terminus with varying tags such as affinity tags and fluorophores for diverse applications including enrichment of NMe2 proteins from a nuclear extract.

A nitrile product can be obtained using selectfluor as oxidizing reagent and NaCN as a nucleophile. Functionalization of the nitrile group on N,N-Me2-Phe-OMe with cysteine methyl ester generate a thiazolidine product. A similar reaction with a peptide N,N-Me2-Phe-Arg-Val and observed the formation of thiazolidine product at the alpha position of the N-terminus. In the case of a peptide containing nitrile-substituted Kme2, Phe-Kme2-Val, demethylation was observed the on reaction with cysteine methyl ester leading to the formation of monomethyl lysine Kme, Phe-Kme-Val. Similar reactions on different Kme2-containing peptides, Phe-Kme2-Val also formed monomethyl lysine Phe-Kme-Val. The same reaction with a peptide, containing both NMe2 and Kme2, N,NMe2-Phe-Kme2-Val using cysteine-methyl ester generated a peptide with a single thiazolidine product at alpha carbon of the N-terminus along with demethylation of the Kme2 to Kme. Overall, the trapping of nitrile with cysteine generated thiazolidine with NMe2 and monomethyl lysine Kme with Kme2. Thus, an N-terminal dimethyl (Nme2) post translational modification can be distinguished from a dimethyl lysine (Kme2) post translational modification.

The stability of the thiazolidine product was examined by incubating the thiazolidine-NMe2-Phe-OMe product in TFA for 24 h. No degradation was observed under the reaction conditions.

Experiments using variant peptides containing reactive amino acids (X=K, H, D, S, M, Y, N, C, R, W) and varying lysine methylation states (Kme1, Kme2 and Kme3) indicate that the reaction is highly chemoselective and generated thiazolidine product is produced with NMe2 only. Oxidation of Met (96%) and some fluorination of Trp (43%) and His (16%) were observed under the reaction conditions.

### Formation of N-Terminal Thiazolidine Labeled Products

A peptide fragment typically the product of N-terminal methyltransferases (NTMT) NMe2-Phe-Gly-Pro-Lys-Arg-Ile-Ala (SEQ ID NO: 2) was expose to reaction conditions using cysteine methyl ester. NMe2-containing peptides were modified to alpha-thiazolidine products, with high conversions >90%. Affinity tag-modified cysteine analogs were synthesized with alkyne, azide, and biotin groups.

### Selective Labeling of NMe2 Peptides in a Complex Cell Lysate Mixture

Experiments were performed to determine the feasibility of labeling low abundant NMe2 peptides in a complex mixture, a cell lysate was spiked with three NMe2 peptides of different amino acid compositions and sizes and incubates with the reaction mixture with Selectfluor™ and NaCN for 2 h followed by the addition of alkyne-cysteine methyl ester for an additional 10 h at room temperature. The reaction mixture was then analyzed by LCMS. The formation of labeled alpha-thiazolidine was observed with all three peptides. No substantial unreacted peptides were detected under the reaction conditions suggesting the robust and chemoselective nature of the reaction for labeling NMe2.

Selective Enrichment of NMe2 Proteins from Nuclear Extract

Nuclear extracts of the prostate cancer cell lysate (LnCap) were incubated with Selectfluor™ and NaCN for 1 h followed by the addition of alkyne-cysteine methyl ester for 10 h. The resulting mixture was filtered using 3K molecular weight cutoff filters to remove all the small byproducts and unreacted starting material. The alkyne-tagged proteins were enriched from concentrated cell lysate using click chemistry with azide-functionalized resin in pull-down experiments. The resin can be washed with solvents to remove any non-covalently bound proteins. The enriched proteins were released from the resin under acidic conditions (95% TFA in water). The analysis of the cell lysate (filtrate) after the enrichment and release of proteins from solid support (eluate) using SDS-PAGE demonstrated the efficient capture and release of NMe2-modified protein thiazolidine.

1. A method of modifying a compound comprising contacting a tertiary amine compound having a N,N′-dimethyl amine group with a cyanide salt and an oxidizing agent under conditions such that a compound having a 2-(methylamino)acetonitrile group is formed in place of N,N′-dimethyl amine.

2. The method of claim 1, wherein the oxidizing agent is 1-chloromethyl-4-fluoro-1,4-diazoniabicyclo[2.2.2]octane bis(tetrafluoroborate) (Selectfluor).

3. The method of claim 1 further comprising contacting the compound having a 2-(methylamino)acetonitrile group with labeled indole providing a N-((1H-indol-3-yl)methyl)-N-methylmethan-1-amine labeled compound.

4. The method of claim 1 further comprising contacting the compound having a 2-(methylamino)acetonitrile group with labeled furan providing N-((furan-2-yl)methyl)-N-methylmethan-1-amine labeled compound.

5. The method of claim 1 further comprising contacting the compound having a 2-(methylamino)acetonitrile group with labeled 1-methyl-1H-pyrrole providing N-((1-methyl-1H-pyrrol-2-yl)methyl)-N-methylmethan-1-amine labeled compound.

6. The method of claim 1 wherein the compound is a peptide having a N,N′-dimethyl lysine amino acid.

7. A method of modifying a compound comprising contacting a tertiary amine compound having a N,N′-dimethyl amine group with a pyridine or pyridine having a para substituted tertiary amine and an oxidizing agent under conditions such that a compound having an aldehyde group is formed in place of N,N′-dimethyl amine.

8. The method of claim 7, wherein the oxidizing agent is 1-chloromethyl-4-fluoro-1,4-diazoniabicyclo[2.2.2]octane bis(tetrafluoroborate) (Selectfluor).

9. The method of claim 7 further comprising contacting the compound having an aldehyde group with labeled hydrazine providing a hydrazine labeled compound.

10. The method of claim 7, wherein the compound is a peptide comprising dimethyl lysine.

11. A method of modifying a peptide comprising
contacting a peptide having an N-terminal N,N′-dimethyl amine group with a cyanide salt and an oxidizing agent under conditions such that a peptide substituted with a nitrile group is formed; and
contacting the peptide substituted with a nitrile group with labeled 2-aminoethane-1-thiol providing a thiazolidine labeled compound.

12. The method of claim 12, wherein the oxidizing agent is 1-chloromethyl-4-fluoro-1,4-diazoniabicyclo[2.2.2]octane bis(tetrafluoroborate) (Selectfluor).

13. The method of claim 12 wherein the label is an alkynyl group providing alkynyl labeled peptide.

14. The method of claim 12 further comprising contacting the alkynyl labeled peptide with a solid surface conjugated to a triazene under conditions such that a triazole peptide is conjugated to the solid surface.

15. The method of claim 14 further comprising contacting the triazole peptide conjugated to the solid surface with an acid solution such that the purified peptide is cleaved from the solid surface providing purified peptide.


SEQUENCE LISTING (2 sequences)

SEQ ID NO: 1 - protein, 5 aa, synthetic construct
GKAKF

SEQ ID NO: 2 - protein, 7 aa, synthetic construct
FGPKRIA